(19) United States
(12) Patent Application Publication
(10) Pub. No.: US 2011/0144029 A1
(43) Pub. Date: Jun. 16, 2011

(54) Model Systems and Materials for the Study and Treatment of Neurodegenerative Diseases

(75) Inventors: Santosh R. D'Mello, Dallas, TX (US); Warren J. Goux, Richardson, TX (US)

(73) Assignee: BOARD OF REGENTS, THE UNIVERSITY OF TEXAS SYSTEM, Austin, TX (US)

(21) Appl. No.: 12/895,763

(22) Filed: Sep. 30, 2010

Related U.S. Application Data
(60) Provisional application No. 61/247,307, filed on Sep. 30, 2009.

(51) Int. Cl. A61K 38/08; C12N 5/07; C12N 5/073; C12Q 1/02; A61P 25/00; A61P 25/28

(52) U.S. Cl. 514/17.8; 435/325; 435/369; 435/29; 514/17.7

(57) ABSTRACT

The present invention provides models for studying the development of, and/or pathologies associated with neurodegenerative diseases, and agents that can modulate such development and/or pathologies.

### CROSS-REFERENCE TO RELATED APPLICATIONS

This application claims priority to U.S. Provisional Application Ser. No. 61/247,307 filed Sep. 30, 2009, the contents of which is incorporated by reference herein in its entirety.

### STATEMENT OF FEDERALLY FUNDED RESEARCH

This invention was made with Government support under Grant No. NS040408 and NS047201 awarded by the National Institutes of Health (NIH) and the National Institute of Neurological Disorders and Stroke (NINDS). The Government has certain rights to this invention.

### TECHNICAL FIELD OF THE INVENTION

The present invention relates in general to the field of models for medical diseases, and more particularly to the development of a cell culture system to understand the mechanisms underlying tau-mediated neurodegeneration and for the screening of therapeutic agents against Alzheimer's disease (AD) and other taupathies.

### INCORPORATION-BY-REFERENCE OF MATERIALS FILED ON COMPACT DISC

None.

### BACKGROUND OF THE INVENTION

Without limiting the scope of the invention, its background is described in connection with protein aggregation in neurodegenerative diseases and models for studying neurodegenerative diseases.

U.S. Pat. No. 6,479,528 issued to Kuret and Khatami (2002) discloses methods for inhibiting and/or reversing tau filament formation or polymerization. These methods can be used for treating certain neurological disorders in vivo by administering pharmaceutical compositions which inhibit and/or reverse tau filament formation or polymerization.

U.S. Pat. No. 6,803,233 issued to Lynch and Bi (2004) provides a model for studying the development of, and/or pathologies associated with neurodegenerative diseases, and agents that can alter such development and/or pathologies. The model described in the '233 patent is especially useful as an Alzheimer's disease model. The model of the invention provides brain cells and a method for increasing neurodegenerative disease characteristics in such cells, especially, induction of neurofibrillary tangles and/or phosphorylated tau and/or tau fragments and/or the production and/or release of cytokines and/or microglia reactions and/or activations and/or inflammation and/or conversion of p35 to p25 and/or the levels and activities of protein kinases by selectively increasing the concentration of cathepsin D to an effective level, and/or by lowering the concentration of cholesterol in such cells. The model also provides a method of reversing such effects, by inhibiting cysteine protease and mitogen activated kinase activity, and especially, by inhibiting calpain, and/or MAP kinase.

European Patent No. 1373529 B1 issued to Van Leuven and Winderickx (2002) relates to a yeast model for the tauopathy as in Alzheimer's disease and other neurodegenerative disorders. The engineered yeast model of the invention can be used in pharmaceutical screening and for modeling neurodegenerative diseases (e.g., Alzheimer's disease, frontotemporal dementia with Parkinsonism) and for in vivo modeling of protein tau biochemistry. The model of the invention can be used as an assay, automated assay or high throughput screening assay for identifying agents, compounds or chemical signals that directly or indirectly affect the biochemistry of tau or tau-protein.

United States Patent Publication No. 20090162336 (Mandelkow et al., 2009) relates to epitopes of the tau protein which are specifically occurring in a phosphorylated state in tau protein from Alzheimer paired helical filaments, to protein kinases which are responsible for the phosphorylation of the amino acids of the tau protein giving rise to said epitopes, and to antibodies specific for said epitopes. The invention further relates to pharmaceutical compositions for the treatment or prevention of Alzheimer's disease, to diagnostic compositions and methods for the detection of Alzheimer's disease and to the use of said epitopes for the generation of antibodies specifically detecting Alzheimer tau protein. Additionally, the invention relates to methods for testing drugs effective in dissolving Alzheimer paired helical filaments or preventing the formation thereof.

Although normally serving to stabilize microtubules, recent evidence suggests that under pathological conditions, tau proteins modified by phosphorylation and proteolytic cleavage promote neurodegeneration. Ectopic expression of tau fragments promotes aggregation of endogenous tau in cultured cells and transgenic mice [9, 45, 46]. Moreover, levels of tau are elevated several fold in AD brains [47, 48]. It has been found that reducing the levels of endogenous tau improves behavioral outcome and is neuroprotective in an AD mouse model [49]. Also consistent with a role for tau as an essential factor in AD-related neurodegeneration is the observation that, tau-deficient primary neurons are resistant to Aβ-induced toxicity [50, 51]. This has led researchers to establish several models in which tau are introduced into primary neurons or cell lines, or cells are exposed to extracellular tau. In some of the studies in which tau was overexpressed in non-neuronal cell lines by transfection, no aggregates or toxicity was reported [64, 65]. In one study by Ding et al. [66] in which normal tau as well as a variety of truncated and point-mutant forms were overexpressed in HEK293 cells, aggregation was reported with some of the truncated and mutant forms but not with wild-type tau. Cell death was not observed with any of the overexpressed constructs. In a tissue culture model utilizing the N2a neuroblastoma cell line, Khlistunova et al. [42] reported that the stable overexpression of truncated forms of tau results in the formation of aggregates and subsequent cell death. In a study by Amadoro et al. [67], truncated forms of tau were overexpressed in cerebellar granule neurons via adenoviral vectors. Such overexpression resulted in the death of the neurons. However, no aggregation of tau or other proteins was reported.

Tau aggregation and toxicity has also been observed by Bandyopadhyay et al. [68] by addition of Congo Red to HEK293 cells stably expressing full-length tau. This study is significant because it recapitulates two key features of Alzheimer disease, viz. aggregation and cell death. However, the model requires the utilization of a synthetic chemical to nucleate tau aggregation [68], and moreover, it utilizes a non-neuronal cell line that is engineered to express highly-elevated levels of tau. In a study performed in the rat pheochromocytoma cell line, Bondareff et al. [69], observed the aggregation of endogenous tau into fibrils. However, the cells had to be differentiated with NGF and then heat-shocked for the formation of fibrils to occur. Cell death was not reported in this model.

Neurotoxicity in SH-SY5Y cells by truncated forms of extracellularly provided tau has been reported by Gomez-Ramos et al. [31]. In this study protein aggregation was not reported although the effect on microtubule organization was described. In another study with extracellularly provided tau to two non-neuronal cell lines, entry of extracellular tau resulted in the aggregation of intracellular tau [70]. However, no cell death was observed.

In summary, although several models of taupathy have been developed recently, with the exception of the report by Khlistunova et al. [42], none display both aggregation and cytotoxicity. Most of the models rely on transfection or infection to introduce tau into the cells. Whether death is selective for neurons or neuronal cell lines has not been studied in these models.

### SUMMARY OF THE INVENTION

The present invention describes two tissue culture based models, utilizing (i) primary neurons, and (ii) a neuroblastoma cell line, for modeling the aggregation of proteins associated with neurodegenerative diseases and the abnormal loss of neurons that defines these diseases. Aggregation and neuronal death is induced by the extracellular delivery of an amyloid forming peptide core made cell permeable by conjugation of a peptide penetrating sequence. In addition to providing model systems to study protein aggregation and neuronal death, the present invention represents model systems that are amenable for high-throughput screening of potential chemical and biological modulators of aggregation and cell death.

The present invention describes the treatment of neurons with a peptide called T-peptide, containing a hexameric sequence from the R₃ region of tau (VQIVYK) (SEQ. ID NO.: 1) fused to, e.g., a nine-residue arginine cell-permeabilizing tag (or similar tag), that recapitulates the aggregation and selective neurotoxicity of tau observed in AD and other taupathies. The tag can be at the amino or carboxy end. In addition, the present invention includes non-covalently bound carrier protein and/or peptide that ferry the hexameric sequence across the cell membrane, e.g., chariot peptide. This novel model system of the present invention can be used to understand the mechanisms underlying tau-mediated toxicity. It can also be used for high-throughput screening of chemical libraries for blockers of tau aggregation or its neurotoxic effect.

The present invention also provides a novel model system for isolating compounds for inhibiting or preventing the development of pathological features of neurodegenerative disease in brain cells. The invention is based on the discovery that T-peptide (a peptide with a hexameric sequence from the tau protein and tagged with a cell-permeabilizing arginine tag) induces the formation of protein aggregates in primary cultures of neurons and a neuronal cell line leading to the degeneration and death of the cell. Aggregation of tau and other proteins and subsequent cell death are characteristic pathological features of neurodegenerative diseases including Alzheimer's disease and various taupathies.

The models described in the present invention offer several advantages over existing models and methods previously described for studying AD and other neurodegenerative conditions. These advantages include: (1). recapitulation of two pathological features of taupathies, protein aggregation and neuronal death, (2). induction by simple addition of a peptide in the cell culture medium, (3). robust and rapid onset of pathological abnormalities, i.e. aggregation is induced within 3 hours and neuronal death, (4). cytotoxicity is selective for neuronal types, and (5). amenability to target and compound screening, and easy adaptation for high-throughput screens.

In one embodiment, the present invention is an in vitro cell culture model or system for studying one or more characteristics that are indicative of one or more taupathies or neurodegenerative conditions in a subject comprising: one or more cells or cell-cultures dispersed in a medium, wherein the one or more cells comprise cerebellar granule neurons, glial cells, neuronal cultures, neurons, kidney fibroblasts, hippocampal cells, human embryonic kidney cells, or any combinations thereof; one or more positive control agents, wherein the positive control agents induce the one or more characteristics that are indicative of one or more taupathies or neurodegenerative conditions in a subjects; one or more negative control agents, wherein the negative control agents do not induce the one or more characteristics that are indicative of one or more taupathies or neurodegenerative conditions in a subjects; one or more test agents, wherein the test agents may induce the one or more characteristics that are indicative of one or more taupathies or neurodegenerative conditions in the subject; and one or more analytical instruments or techniques that detect the one or more characteristics that are indicative of one or more taupathies or neurodegenerative conditions in the subject, wherein a comparison of the test agents versus the positive and negative controls is indicative of having an effect on the cells. In one aspect, the one or more taupathies or neurodegenerative conditions are selected from the group consisting of Alzheimer's disease (AD), Parkinson's disease, Huntington's disease, frontotemporal dementia, progressive supranuclear palsy, corticobasal degeneration, and frontotemporal lobar degeneration (Pick's disease). In another aspect, the characteristics evaluated comprise formation of one or more neurofibrillary tangles (NFTs), tau protein aggregation, tau protein hyperphosphorylation, tau protein cleavage or any combinations thereof. In another aspect, the one or more positive and negative control agents comprise one or more peptides, proteins, drugs, biological agents, chemical agents or any combinations thereof. In another aspect, the one or more positive control peptides comprise an amino acid sequence VQIVYK (SEQ. ID NO.: 1), VQVVYK (SEQ. ID NO.: 6), VQIVFK (SEQ. ID NO.: 7) or any combinations thereof. In another aspect, the one or more negative control peptides comprise an amino acid sequence VQIVKK (SEQ. ID NO.: 4), VQVVVK (SEQ. ID NO.: 5), or any combinations thereof. In another aspect, the one or more test agents comprise one or more peptides, proteins, drugs, biological agents, chemical agents or any combinations thereof. In another aspect, the one or more test agents are synthesized artificially, occurs naturally, or is isolated from the subject. In yet another aspect, the subject is selected from the group consisting of a human subject, a rodent, a mouse, and a mammalian subject. In another aspect, the one or more analytical instruments or techniques comprise staining techniques, microscopy, aggregation kinetic analysis, immunochemical and Western Blot analysis, cell viability analysis, or any combinations thereof.

In another embodiment, the invitation is an in vitro cell culture method for detection of one or more characteristics indicative of Alzheimer's Disease (AD) and other taupathies in a subject suspected of having AD or the other taupathies comprising the steps of: providing one or more cells or cell-cultures dispersed in a medium, wherein the one or more cells or cell cultures comprise cerebellar granule neurons, glial cells, neuronal cultures, neurons, kidney fibroblasts, hippocampal cells, human embryonic kidney cells, or any combinations thereof; providing one or more positive control agents, wherein the positive control agents induce the one or more characteristics that are indicative of AD and other taupathies; providing one or more negative control agents, wherein the negative control agents do not induce the one or more characteristics that are indicative of AD and other taupathies; obtaining a test sample from the subject suspected of having AD or the other taupathies, wherein the test sample is an isolated protein, a peptide, a biological sample, a tissue sample or any combinations thereof; incubating the test sample, the positive control agent, and the negative control agent with the one or more cells or cell culture; and detecting one or more characteristics indicative of AD and other taupathies in the one or more cells or cell culture following the incubation by a combination of staining techniques, microscopy, aggregation kinetic analysis, immunochemical and Western Blot analysis, and cell viability analysis. In another aspect, the presence of one or more neurofibrillary tangles (NFTs), tau protein aggregation, tau protein hyperphosphorylation, tau protein cleavage is indicative of AD and other taupathies.

In another embodiment, the invention includes a method for screening and testing an activity of one or more agents against AD and other taupathies comprising the steps of: providing one or more cells or cell-cultures dispersed in a medium, wherein the one or more cells or cell cultures comprise cerebellar granule neurons, glial cells, neuronal cultures, neurons, kidney fibroblasts, hippocampal cells, human embryonic kidney cells, or any combinations thereof; providing one or more positive control agents, wherein the positive control agents induce the one or more characteristics that are indicative of AD and other taupathies; providing one or more negative control agents, wherein the negative control agents do not induce the one or more characteristics that are indicative of AD and other taupathies; providing one or more test agents against AD or the other taupathies, wherein the test agents comprise naturally occurring, synthesized or isolated proteins, peptide, chemical and biological substances or any combinations thereof; incubating the test agent, the positive control agent, and the negative control agent with the one or more cells or cell culture; and detecting one or more characteristics indicative of AD and other taupathies in the one or more cells or cell culture following the incubation by a combination of staining techniques, microscopy, aggregation kinetic analysis, immunochemical and Western Blot analysis, and cell viability analysis. In another aspect, the one or more positive control peptides comprise an amino acid sequence VQIVYK (SEQ. ID NO.: 1), VQVVYK (SEQ. ID NO.: 6), VQIVFK (SEQ. ID NO.: 7) VQVVYK-R9 (V-peptide) (SEQ. ID NO.: 6), VQVVVK-R9 (VV-peptide) (SEQ. ID NO.: 5), VQIVKK-R9 (K-peptide) (SEQ. ID NO.: 4), and VQIVFK-R9 (F-peptide) (SEQ. ID NO.: 7), or any combinations thereof.

In another embodiment, the invention includes a pharmaceutical composition for treating AD or other taupathies in a subject comprising: one or more active agents dispersed in a pharmaceutical carrier, wherein the one or more active agents inhibit a formation of one or more neurofibrillary tangles (NFTs) and inhibit tau protein aggregation, hyperphosphorylation, and cleavage in the subject; and one or more optional excipients, fillers, diluents, extended or controlled release agents, bulking agents, antiadherents, binders, lubricants, preservatives or any combinations thereof. In another aspect, the composition is administered subcutaneously, intravenously, peritoneally, orally, and intramuscularly.

Yet another embodiment includes a method for treating AD and other taupathies in a subject comprising the steps of: identifying the subject in need for treatment against AD and other taupathies; and administering a pharmaceutical composition comprising one or more active agents dispersed in a pharmaceutical carrier, wherein the one or more active agents inhibit a formation of one or more neurofibrillary tangles (NFTs) and inhibit tau protein aggregation, hyperphosphorylation, and cleavage in the subject. In another aspect, the method further comprising the steps of: monitoring the inhibition of the formation one or more neurofibrillary tangles (NFTs) and tau protein aggregation, hyperphosphorylation, and cleavage in the subject; and administering the pharmaceutical composition repeatedly if necessary until the formation of one or more neurofibrillary tangles (NFTs) and tau protein aggregation, hyperphosphorylation, and cleavage in the subject is halted.

The present invention also includes a composition for treating taupathies or neurodegenerative conditions in a subject comprising: a fusion peptide comprising an amyloid forming peptide and a cytoplasmic penetration peptide, wherein the fusion protein triggers cell death. In another aspect, the fusion peptide is selected from the group consisting of Ac-vqivyk-R₉—NH₂ (lower case amino acid codes denote D-amino acids) (SEQ. ID NO.: 1), Ac-vqivyk-NH₂(Core peptide) (SEQ. ID NO.: 1), Ac-vqvvyk-R₉—NH₂ (V-peptide) (SEQ. ID NO.: 6), Ac-VQIVYKR₉—NH₂ (T (L)-peptide) (SEQ. ID NO.: 1), and Ac-vqivfk-R₉—NH₂ (F-peptide) (SEQ. ID NO.: 7). In yet another aspect, the fusion peptide is selected from Ac-vqvvvkR₉—NH₂ (VV-peptide) (SEQ. ID NO.: 5) and Ac-vqivkk-R₉—NH₂ (K-peptide) (SEQ. ID NO.: 4). Although the above sequences are listed as D-amino acids, the L-amino acid can also be used or a mixture thereof. In another aspect, the one or more taupathies or neurodegenerative conditions are selected from the group consisting of Alzheimer's disease (AD), Parkinson's disease, Huntington's disease, frontotemporal dementia, progressive supranuclear palsy, corticobasal degeneration, and frontotemporal lobar degeneration (Pick's disease).

Another embodiment of the present invention includes an in vitro cell culture model or system for studying one or more characteristics that are indicative of one or more taupathies or neurodegenerative conditions in a subject comprising: one or more cells or cell-cultures dispersed in a medium, wherein the one or more cells comprise cerebellar granule neurons, glial cells, neuronal cultures, neurons, kidney fibroblasts, hippocampal cells, human embryonic kidney cells, or any combinations thereof; a fusion peptide comprising an amyloid forming peptide and a cytoplasmic penetration peptide, wherein the fusion protein triggers aggregation and neuronal cell death; and one or more test agents, wherein the test agents may cause survival of the neuronal cells by the fusion peptide. In addition, a non-covalently bound peptide may be used to transport the amyloid forming peptide across the cell membrane. In one aspect, the one or more taupathies or neurodegenerative conditions are selected from the group consisting of Alzheimer's disease (AD), Parkinson's disease (PD), Huntington's disease (HD), frontotemporal dementia, progressive supranuclear palsy, corticobasal degeneration, and frontotemporal lobar degeneration (Pick's disease). In another aspect, the fusion protein comprises a fusion peptide selected from VQVVYK-R₉ (V-peptide) (SEQ. ID NO.: 6), VQVVVK-R₉ (VV-peptide) (SEQ. ID NO.: 5), and VQIVFK-R₉ (F-peptide) (SEQ. ID NO.: 7), or any combinations thereof.

### BRIEF DESCRIPTION OF THE DRAWINGS

For a more complete understanding of the features and advantages of the present invention, reference is now made to the detailed description of the invention along with the accompanying figures and in which:

FIG. 1 shows the aggregation kinetics of peptides studied in vitro. Aggregation kinetics of T-peptide (filled triangles), V-peptide (filled diamonds), F-peptide (X), Core peptide (open triangles), VV-peptide (open diamonds) and K-peptide (filled circles). FIG. 1 shows the time dependence of the fluorescence intensity for 10 μM peptide, 33 μM heparin and 100 μM ThS in 5 mM MOPS, pH 7.2, containing 0.15 M NaCl (Buffer A). Solid lines represent fitted values;

FIG. 2 shows the TEM of peptides studied: TEM of (2A) Core peptide, (2B) T-peptide (arrows denote helical twist), (2C) V-peptide, (2D) K-peptide. All peptides were prepared at 100 μM in Buffer A and stained with uranyl acetate;

FIG. 3 shows the effects tau peptides on survival of cerebellar granule neurons. Neuronal cultures were switched to serum-free medium T-peptide, K-peptide, Core-peptide, V-peptide. VV-peptide, and T (L)-peptide. All peptides were used at 25 μM concentration. Phase contrast micrographs show appearance of the cultures after 24 h of peptide treatment. The Live/Dead viability assay was also performed. Nuclei of dead cells are stained red whereas cytoplasm of living cells are stained green. In the case of sick and dying cells, green staining is condensed;

FIG. 4 shows neuronal cell viability following treatment with tau peptides. Cerebellar granule neurons were switched to serum-free medium containing various concentrations of T-peptide, K-peptide, Core-peptide, V-peptide, VV-peptide, and T (L)-peptide. Cell viability was measured using two independent methods 24 h after peptide treatment and results are graphed. * indicates p<0.05 compared with control cells that were treated with HK: (4A) Results from Red/Green viability assay, (4B) Results from MTT assay;

FIG. 5 shows the mechanism of neurotoxicity by T-peptide that is neither fully apoptotic nor autophagic: (5A) Nuclear morphology of cerebellar granule neurons treated with T-peptide. Cerebellar granule neuron cultures were treated with T-peptide in medium containing depolarizing levels of potassium (HK). The cultures were then fixed and stained with DAPI to visualize nuclear morphology. Sister cultures were treated for 24 hours with HK alone (in which they survive) or with non-depolarizing medium (5 mM KCl or LK) in which they undergo apoptosis. In contrast to the nuclear condensation and fragmentation (marked with asterisks) in LK-treated cultures, nuclei of cultures treated with T-peptide displayed no fragmentation. Large bright spots are visible in the nuclei of T-peptide treated neurons, (5B) LC3 cleavage pattern following T-peptide treatment of neurons: Cerebellar granule neurons were either untreated (Unt) or treated with K peptide for 3 h (K3) or with T peptide for 1 and 3 h (T1 and T3, respectively). Total cell lysates were prepared and subjected to Western blotting using an LC3 antibody. While the 18 kDa cytoplasmic LC-1 could be clearly detected, only a faint 16 kDa band representing the autophagosome-associated LC-II form of the LC3 protein could be detected. No alterations in the pattern of LCI and LCII were discernible. Lysates from cells treated with K and T peptide for 6 and 9 h were also analyzed but yielded similar results (not shown);

FIG. 6 shows the selective vulnerability of neurons to T-peptide toxicity. Primary cultures and cell lines were treated in serum-free medium with T-peptide for 24 hours. Viability was quantified using the Live/Dead assay. * indicates p<0.05 compared with control cells that were treated with serum-free medium without peptide: (6A) Results from the analyses of cerebellar granule neurons, cortical neurons, and kidney fibroblasts cultured from rats, (6B) Results from the HT22 and HEK293 cell lines;

FIG. 7 shows the subcellular localization of T-peptide. HT-22 cells were treated with a FITC-tagged form of T-peptide (T-FITC-peptide) for 3 h. Subcellular localization was visualized by fluorescence microscopy. The cells were also counterstained with DAPI (for nuclear morphology), and propidium iodide (PI, for cell viability). Note intense and punctate staining in the nucleus. In comparison, cytoplasmic staining is generally diffuse.

FIG. 8 shows the aggregation of the T-peptide forms within the nuclei. Thioflavin-staining of T-peptide treated cells. HT22 cells were treated with T-peptide, K-peptide, and V-peptide. 3 hours later the cultures were stained with Thioflavin-S (for aggregates), DAPI (for nuclear morphology), and propidium iodide (PI, for cell viability). While staining is easily detectable in T-peptide and V-peptide treated cultures, it is not detectable in cultures treated with K-peptide;

FIG. 9 shows that the phosphorylation of T-peptide is not required for neurotoxicity. Cerebellar granule neurons were treated with different concentrations of F-peptide for 24 h, after which cell viability was quantified using the Red/Green viability assay. F-peptide was synthesized using L-amino acids. * indicates p<0.05 compared with control cells that were treated with HK

FIG. 10A-10D illustrate the elevated level of intracellular tau does not increase neuronal vulnerability to T-peptide. FIG. 10A is an image of the viability in cerebellar granule neurons. Transfected neuronal cultures were switched to HK medium containing no additives (NA), 10 μM K-peptide, or 10 T-μM peptide. Cell viability was quantified 24 h later and normalized to viability of GFP-transfected neurons in HK. FIG. 10B is an image of the viability in HT22 cells. Cultures were either untreated (Un) or treated with 25 μM K-peptide or 25 μM T-peptide. Cell viability was quantified 24 h later and normalized to viability of GFP-transfected untreated cultures. FIG. 10C is an image of the localization of tau-YFP in healthy and dead neurons treated with K-peptide or T-peptide. FIG. 10D is an image of the localization of tau-YFP in healthy and dead neurons treated with K-peptide or T-peptide.

FIG. 11 is an image of the expression of endogenous tau is reduced by T-peptide. Cerebellar granule neurons were treated for 3 h or 6 h with HK medium containing no additives (NA) or 10 μM each of Core-peptide (C), T-peptide (T), K-peptide (K). Cell lysates were prepared and subjected to Western blot analysis using antibodies against total tau, Ser262-phospho-tau, and Ser462-phospho-tau. The blot was also probed with tubulin antibody to verify that similar amounts of protein were loaded in each lane.

FIG. 12 is an image of the effects of tau peptides on the mitochondrial membrane potential of HT-22 cells. HT-22 cells were treated with various concentrations of T-peptide (♦), K-peptide (▴), Core-peptide (x) or T (L)-peptide (▪) for 24 hours in serum-free medium. The mitochondrial membrane potential of the cells was measured using the fluorescent dye, JC-1. Cells were labeled with JC-1 solution for 2 hours at 37° C. Samples were imaged using a fluorescent plate reader set to detect both red and green fluorescence. The ratio of the red to green fluorescence units is a measure of the mitochondrial membrane potential. *indicates p<0.05 compared with non-treated control cells.

FIG. 13 is an image of the non-tau related aggregating peptides are not neurotoxic.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention describes a cell culture model of taupathy using a simple hexameric peptide with the sequence ³⁰⁶VQIVYK³¹¹ (SEQ. ID NO.: 1) located within the third microtubule-binding repeat of tau, rendered cell-permeable by a tag of nine arginine residues (R₉). This peptide (VQIVYK-R₉) (SEQ. ID NO.: 1), designated as T-peptide, can self-assemble in vitro into paired-helical filament-like aggregates. The model system described here represents a convenient paradigm to study the mechanisms underlying tau aggregation and neurotoxicity, and to identify compounds that can prevent these effects. Such compounds have value in the treatment of Alzheimer's diseases and other taupathies.

The present invention further provides a method for triggering brain cells or cell-lines resembling brain cells to develop pathological features of brain cells from a brain that is afflicted from neurodegenerative disease. A novel feature of the invention is the identification of a trigger peptide that can induce protein aggregation and brain cell degeneration when introduced into brain cells. The system uses as a trigger an amyloid forming peptide core consisting of 2-20 amino acids conjugated to a tag peptide sequence capable of making the entire peptide-trigger membrane-permeable. In addition, the core peptide to be ferried may be or include a non-covalent associated transporting peptide or protein The core sequences may include tau nucleating sequences ³⁰⁶VQIVYK³¹¹ (references from full length tau) (SEQ. ID NO.: 1), ²⁷⁵VQIINK²⁸⁰ (SEQ. ID NO.: 2), the Aβ sequence ¹⁶KLVFFA²¹ (SEQ. ID NO.: 3), ³⁷⁵KLTFR³⁷⁹ (SEQ. ID NO.: 8), mutants of these sequences or other sequences which may act as amyloidogenic seeds for aggregation or other proteins known to those skilled in the art [15]. The tag peptide may include poly-arginine based sequences, the third helix of the homeodomain of Antennapedia protein, HIV Tat peptide or other peptide sequences able to make substances membrane permeable [71]. The present invention also provides a novel model system for isolating compounds for inhibiting or preventing the development of pathological features of neurodegenerative disease in brain cells. The invention is based on the discovery that T-peptide (a peptide with a hexameric sequence from the tau protein and tagged with a cell-permeabilizing arginine tag) induces the formation of protein aggregates in primary cultures of neurons and a neuronal cell line leading to the degeneration and death of the cell. Aggregation of tau and other proteins leading to cell death are characteristic pathological features of neurodegenerative diseases including Alzheimer's disease and various taupathies.

The models described in the present invention offer several advantages over existing models and methods previously described for studying AD and other neurodegenerative conditions. These advantages include: (1). Recapitulation of two pathological features of taupathies, protein aggregation and neuronal death, (2). Induction by simple addition of a peptide in the cell culture medium, (3). Robust and rapid onset of pathological abnormalities, i.e. aggregation is induced within 3 hours and neuronal death, (4). Cytotoxicity is selective for neuronal types, and (5). Amenability to target and compound screening, and easy adaptation for high-throughput screens.

Embodiments of the invention includes methods comprising the steps of culturing of brain cells; synthesis of a cell-permeable peptide by the attachment of a poly-arginine tag; contacting the brain cells with the peptide that increases protein aggregation and cell death; and identification of a hexameric sequence of tau with self-aggregating and neurotoxic action. Another embodiment of the present invention is a method for increasing protein aggregation in brain cells followed by treatment of the brain cells with pharmacological agents to reduce or prevent cell death.

The method of the present invention uses either neurons (cerebellar granule neurons and cortical neurons) and/or the HT22 neuroblastoma cell line. The peptide of the present invention comprises a hexameric core sequence that induces protein aggregation and neuronal death. In one example, the cell-permeability of the peptide is made possible by the conjugation of a poly-arginine tag to the hexameric sequence.

The method of the present invention can also be used to screen for pharmacological agents and biological factors that slow down, reduce, or prevent death of neurons or neuronal cell lines, e.g., pharmacological inhibitors of JNK, such as SP600125 represents one such agent. The method can further be used to screen for modulators of the aggregation process by (i) staining with thioflavin-S and (ii) using FITC-conjugated T-peptide, and evaluating the presence of aggregates. The method can also used to screen for inhibitors of neuronal death by (i) using the live/dead assay and (ii) the MTT assay.

Intracellular aggregation of the microtubule-associated protein tau is a pathological hallmark in many neurodegenerative diseases including Alzheimer's disease, frontotemporal dementia, and other taupathies. Despite effort by numerous laboratories, the mechanisms underlying tau aggregation in neurons and the role these aggregates play in neuronal death has remained controversial. When cultures of primary neurons are treated with T-peptide of the present invention they die within 24 hours. Neurodegeneration correlates with the ability of the peptide to aggregate. Two peptides with mutations in the hexameric core, K-peptide (VQIVKK) (SEQ. ID NO.: 4) and VV-peptide (VQVVVK) (SEQ. ID NO.: 5), that are incapable of aggregating are not toxic, whereas two other mutant peptide, V-peptide (VQVVYK) (SEQ. ID NO.: 6) and F-peptide (VQIVFK) (SEQ. ID NO.: 7), which aggregate are also neurotoxic. Furthermore, non-tau related peptides which aggregate, including STVIIE and SQIVYA, are not toxic. Within neurons, T-peptide localizes predominantly within the nucleus in aggregates. Although localizing to the nucleus, treatment of neurons with T-peptide induces aggregation of cellular proteins in the cytoplasm as judged by positive thioflavin-S staining In comparison to neurons, non-neuronal cells are less sensitive to T-peptide-induced toxicity, recapitulating in part, the selective loss of neurons in taupathies. Thus, the cell-culture model of the present invention represents a convenient paradigm to study the mechanisms underlying tau aggregation and neurotoxicity, and to identify compounds that can prevent these effects.

A major pathological hallmark of Alzheimer's disease (AD) and other taupathies is the formation of neurofibrillary tangles (NFTs) within neurons of the brain [1-3]. NFTs are composed of insoluble twisted filaments, known as paired helical filaments (PHFs) made up of aggregated tau, a microtubule-associated protein. Because the appearance of NFTs correlates with neuronal death and loss of cognitive function in AD, it is generally believed that aggregated tau has a toxic effect on neurons.

Tau is a protein predominantly expressed in neurons where it functions to promote tubulin polymerization and microtubule stability. Under normal circumstances tau is a highly soluble protein that does not tend to aggregate. Tau's aggregation into filaments in neurodegenerative pathologies is accompanied by abnormal posttranslational modifications, the best studied of which is hyperphosphorylation [4]. Another modification gaining considerable attention is cleavage of tau by proteases including caspases and calpains. Indeed, tau cleavage has been observed in AD as well as in experimental models of neurodegeneration [5-7]. Although there is agreement that tau assembles into PHFs, is phosphorylated, and can be proteolyzed, the relationship between these different modifications and their significance to neurodegeneration remains unclear and a topic of much disagreement. For example, while it is generally felt that hyperphosphorylation of tau promotes its aggregation, emerging evidence suggests that tau phosphorylation actually inhibits its aggregation [8, 9]. Other studies have suggested that aggregation of tau into PHFs is not directly neurotoxic [10], or that formation of tau filaments protects against the neurotoxic effects of hyperphosphorylated tau [11]. More recently, smaller pre-fibrillar tau aggregates or soluble tau monomers have been suggested to be the toxic species [12-14].

Work performed both in vitro and in cultured cells has shown that proteolytic fragments of tau containing the microtubule-binding repeat regions aggregate robustly forming AD-like PHFs [15, 16]. Aggregated fragments of tau are able to nucleate the aggregation of full-length tau both in cultured cells and in transgenic mice [9, 17, 18]. A six amino acid region, ³⁰⁶VQIVYK³¹¹, (SEQ. ID NO.: 1) located within the third microtubule-binding repeat of tau has been identified as the smallest sequence of tau capable of self-assembly into pathological PHFs [15]. The inventors and others have previously shown that this hexapeptide has a very high propensity for self-aggregation [15, 19, 20]. Deletion of this six amino acid sequence from full-length tau dramatically reduces the ability of tau to aggregate in transfected cells [21].

Since the hexameric motif can self-aggregate and promote assembly of full-length tau into PHF-like filaments, the inventors tested the intracellular delivery of this hexapeptide to determine if it would be sufficient to induce neurotoxicity. The inventors found that the VQIVYK (SEQ. ID NO.: 1) sequence is in fact, a potent inducer of neuronal death. Its ability to kill non-neuronal cells is substantially lower, recapitulating the selective loss of neurons in AD and other neurodegenerative taupathies.

Unless mentioned otherwise, all chemicals were purchased from Sigma-Aldrich (St. Louis, Mo., USA). Tissue culture reagents including cell culture medium, fetal calf serum (FCS), poly-L-lysine, L-glutamine, penicillin-streptomycin, and gentamycin were from Invitrogen (Carlsbad, Calif., USA). LC3 antibody was from Sigma-Aldrich. Fluorescence-conjugated secondary antibodies were from Jackson ImmunoResearch Laboratories, Inc (West Grove, Pa.).

Peptides: N-acetyl peptide amides Ac-vqivyk-R₉—NH₂ (T-peptide; lower case amino acid codes denote D-amino acids) (SEQ. ID NO.: 1), Ac-vqivyk-NH₂ (Core peptide) (SEQ. ID NO.: 1), Ac-vqvvyk-R₉—NH₂ (V-peptide) (SEQ. ID NO.: 6), Ac-vqvvvkR₉—NH₂ (VV-peptide) (SEQ. ID NO.: 5), Ac-vqivkk-R₉—NH₂ (K-peptide) (SEQ. ID NO.: 4), Ac-VQIVYKR₉—NH₂ (T (L)-peptide) (SEQ. ID NO.: 1), Ac-VQIINK-R9 (SEQ. ID NO.: 2), Ac-KLTFR-R9 (SEQ. ID NO.: 8), and Ac-VQIVFK-R₉—NH2 (F-peptide) (SEQ. ID NO.: 7) were prepared by solid-phase peptide synthetic methods according to previously published procedures and purified by reverse-phase HPLC using a water-acetonitrile gradient {{54 Rojas Quijano, F. A. 2006; 56 Goux, W. J. 2004}}. An FITC-labeled version of T-peptide (FITC-T-peptide) was prepared from a resin bound T-peptide having two N-terminal amino hexanoic “linker residues” on to which 5′(6)-carboxyfluorescein was coupled using standard solid phase methodology. Peptide purity was checked using MALDI-TOF MS. Stock solutions of the peptides were prepared (˜1 mg/mL) in deionized water. Final concentrations determined by weight, assuming the TFA salt of the purified peptide, agreed within 5% of concentrations determined using the reported molar extinction coefficients for tyrosine [ε₂₇₆ of 1390 M⁻¹cm⁻¹ or for the peptide bond [ε₁₉₂ of 7110 M⁻¹cm⁻¹. Prior to TEM and kinetics measurements the stock solutions were filtered through a Millipore Ultrafree-MC 100,000 NMWL filter unit (Billerica, Mass.) by centrifuging for 10 minutes at 14,500 rpm, using an Eppendorf MiniSpin plus bench top centrifuge, in order to eliminate aggregates.

Transmission Electron Microscopy (TEM): Filtered stock peptide solutions were diluted to 100 μM in 5 mM MOPS containing 0.1% NaN₃, 0.15 M NaCl, pH 7.2 (Buffer A) and were incubated at room temperature overnight. Samples were loaded onto carbon-coated Formvar copper grids (200 mesh) and stained with 2% wt/wt uranyl acetate. A JEOL 1200 EX scope interfaced to a digital camera was used to visualize samples.

Kinetic analysis: Polymerization kinetics were measured by monitoring the increase in Thioflavin S (ThS) fluorescence at 490 nm (440 nm excitation). Slit widths were 15 and 20 nm respectively for excitation and emission. All measurements were carried out at 21° C. with a Perkin Elmer LS50B spectrofluorimeter using a 75 μL quartz cell. Data points were collected at 6 s intervals over the course of the experiment (60 min). In order to initiate polymerization a freshly filtered stock solution was diluted to 10 μM in 200 μL of Buffer A containing 33 μM heparin (Sigma-Aldrich; 3,000 MW) and 100 μM Thioflavin S (ThS). Kinetics data, corrected for the blank, were fit using a non-linear least squares algorithm to a Gompertz-like growth curve {{260 Necula, M. 2004}} (Eqn. [1])

$$y = {e}^{( - {k}^{′} t )} {Ae}^{- {e}^{( \frac{( t - {t}_{i} )}{b} )}} \qquad [ 1 ]$$

where y is defined as the fluorescence signal at time t, tᵢ corresponds to the inflection point of the curve, or the time of maximum growth rate, A is the maximum fluorescence observed for a given sample, and b=1/k, where k is the rate constant of aggregation, in units of s⁻¹. Lag-times were calculated as tᵢ−b. The pre-growth curve exponential, e^{(-k′t)} was inserted to take into account the slow decrease in fluorescent intensity resulting from ThS bleaching, quenching and gravitational precipitation of the amyloid-ThS complex. Standard errors in fitted parameters were estimated assuming a 10% error in the sum of the squares.

Culturing and treatment of neurons: Cerebellar granule neurons were cultured from 7-8 day old rats as previously described [22]. The neurons were plated in 24-well with poly-L-lysine coated glass coverslips at a density of ˜1 million cells/well in Basal Eagle's Medium with Earle's salts (BME) supplemented with 10% FCS, 25 mM KCl, 2 mM glutamine and 100 mg/ml gentamicin. To prevent mitosis of glial cells, cytosine arabinofuranoside (10 mM) was added to culture 16-20 hrs after plating. Neuronal cultures were treated with peptides 6-7 days after plating. For treatment, the cultures were switched to serum-free medium containing 25 mM KCl (referred to as high potassium or HK medium). Although serum is required for proper maturation, after 5 days in culture, the neurons survive normally in the absence of serum provided depolarizing levels of KCl is added [22].

Cultures of kidney fibroblasts and treatment with peptides: Kidneys were taken for 7-8 day old rats and placed in Dulbecco's Modified Eagle's Medium (DMEM). They were then diced and trypsinized for 8-12 mins. Trypsin was inactivated with trypsin inhibitor and centrifuged at 500 g for 1 min. Pellets were resuspended in DMEM and broken-up with a pasteur pipette 25 times, then centrifuged at 1000 g rpm for 1 min. Again the pellet was resuspended in DMEM, broken-up with Pasteur pipette, and centrifuged at 1000 g rpm. After resuspending pellets in DMEM, the cells were centrifuged again at 500 g for 10 min. The cell pellet was then dissociated and plated in DMEM supplemented with 10% FCS, 50 U/ml penicillin and 50 μg/ml streptomycin in 24-well dishes. Treatment with peptides was done at 40-50% confluency for Live/Dead assay and 80-90% confluency for MTT assay.

HT-22 and HEK293 cell culture: HT-22 cells were maintained in DMEM supplemented with 10% FCS, 50 U/ml penicillin and 50 μg/ml streptomycin. HEK293 were maintained in DMEM supplemented with 10% FCS, 50 U/ml penicillin and 50 μg/ml streptomycin. Both cell lines were treated with peptide at 70-80% confluency in serum-free media.

Cell viability quantification: Cell viability was quantified using the Live/Dead assays with a commercially available kit (Invitrogen). This is a two-color fluorescence assay utilizing two cell-permeable compounds, calcein acetoxymethyl (calcein AM) and ethidium homodimer-1 (EthD-1). Live cells have intracellular esterases that convert non-fluorescent calcein AM to the intensely green fluorescent calcein, which is retained within the cytoplasm of cells. Because dead cells have damaged membranes, EthD-1 enters and is fluorescent when bound to nucleic acids. EthD-1 stains the nuclei of damaged or dead cells. As recommended by the manufacturer, cells plated in wells with cover-slips were incubated in Live/Dead reagent (1 uM calcein AM and 4 uM EthD-1) for 30 min, and then mounted on slides. Cell viability was also analyzed using the MTT [3-(4,5-Dimethylthiazol-2-yl)-2,5-diphenyltetrazolium bromide] assay based on the evaluation of mitochondrial function. MTT assays were performed as previously described [23]. Briefly, MTT (5 mg/ml in culture medium) was added to the culture medium and the cells incubated at 37° C. for 1 h. The assay was stopped by adding lysis buffer [20% sodium dodecyl sulfate (SDS) in 50% N,N-dimethylformamide, pH 4.7]. Absorbance was measured spectrophotometrically at 570 nm after an overnight incubation at room temperature.

Western blot analysis: Cells were washed once with ice-cold phosphate-buffered saline (PBS) and lysed using lysis buffer (1% Triton, 20 mM Tris-HCl [pH 7.5], 150 mM NaCl, 1 mM sodium EDTA, 1 mM EGTA, 2.5 mM sodium pyrophosphate, 1 mM beta-glycerophosphate, 1 mM Na₃VO₄, 1 μg/ml leupeptin, and one protease inhibitor tablet). Protein concentration was measured with Bradford assay reagent (Bio-Rad). Protein concentration was normalized to 50-75 ug and 6×SDS was added to each sample. Samples were ran on 15% denaturing gels and transferred to PVDF membrane and then blocked for 1 hour and incubated for LC3 at 1:1000 dilution. After washes, the membrane was incubated with horseradish peroxidase-conjugated secondary antibody. Following the incubation, membranes were washed and developed with Enhanced Chemiluminescence reagent (Amersham Biosciences).

Staining of tau aggregates: Aggregates were stained with thioflavin-S and counter-stained with propidium Iodide (PI) for dead cells and 4′,6-diamidino-2-phenylindole (DAPI) for the nucleus of all cells. 1-3 hours after treatment with peptide, cells were incubated in 20 μg/ml of PI contained in media for 10 min, washed three times with media, incubated in cold 4% paraformaldehyde for 10 min, washed with PBS, incubated in 0.1% triton-X for 5 min, incubated in DAPI for 10 min and washed with PBS. Improved thioflavin-S staining technique was employed (Bing et al., 2002). Briefly, the fixed cells were incubated in 0.01% thioflavin-S, washed three times with 70% ethanol for 5 min each, washed with water, incubated in 0.25% KMnO4 for 4 min, washed with water, incubated in 1% NaBH4 for 4 min, and then again washed with water. Cells were mounted on slides using Fluoromount-G mounting media (SouthernBiotech, Birmingham, Ala.).

Analysis of viability data: Unless indicated otherwise, data are given as mean+standard deviation. For statistical significance, data were evaluated at a 0.05 level of significance with the Bonferroni ANOVA.

Generation and aggregation analysis of tau peptides: Previous studies have shown that tau polymerization involves the formation of a β-sheet structure arising from short hexapeptide motifs in the second and third repeat of tau, specifically ²⁷⁵VQIINK²⁸⁰ (SEQ. ID NO.: 2) in microtubule-binding repeat R2 and ³⁰⁶VQIVYK³¹¹ (SEQ. ID NO.: 1) in repeat R3 [15, 24]. The inventors and others have previously shown that the R3 hexapeptide, Ac-³⁰⁶VQIVYK³¹¹-NH₂ (SEQ. ID NO.: 1) could, by itself, self-nucleate and form fibrils in vitro [19, 20, 25]. To render this hexapeptide cell-permeable, the inventors attached a HIV Tat-like motif composed of nine arginine residues (R₉) to the C-terminal. Arginine-rich peptides translocate the plasma membrane efficiently and are used for intracellular delivery of macromolecules and chemicals [26-29]. The hexameric core was synthesized with D-amino acids, while the R₉ tag was composed of L-arginine. The rationale for synthesizing a D-core was two-fold; first, it would be more stable against proteolytic degradation, and second, the D-version would not bind to specific protein targets within the cell thus allowing a better evaluation of the effect of aggregation itself on cell survival. The vqivyk-R₉ peptide was designated as T-peptide. Neurotoxicity studies were also carried out on the T-peptide lacking the R₉ tag, vqivyk (SEQ. ID NO.: 1), which we designate as the Core-peptide. Mutants of the T-peptide, vqvvyk-R₉ (V-peptide) (SEQ. ID NO.: 6), vqvvvk-R₉ (VV-peptide) (SEQ. ID NO.: 5), vqivkk-R₉ (K-peptide) (SEQ. ID NO.: 4), and VQIVFK-R₉ (F-peptide) (SEQ. ID NO.: 7), were also studied to determine if toxicity was a function of primary structure and the ability of these peptides to self-associate into aggregates. Table 1 summarizes the structures and abbreviations for the peptides used in the study.

TABLE 1
Summary of structural and kinetic data for the peptides studied.

| | | | | Fitted kinetic parameters^{b} | | |
|---|---|---|---|---|---|---|
| | | | | | k | k′ |
| | | Expected | Observed | | (s⁻¹) × | (s⁻¹) × |
| Peptide | Sequence^{a} | MW | MW | A | 10³ | 10⁵ |
| Core-peptide | Ac-vqivyk-NH₂ | 790 | 792 | 6.3 | 10.6 | — |
| T-peptide | Ac-vqivyk-R₉-NH₂ | 2195 | 2195 | 9.1 | 8.5 | 4.4 |
| T(L)-peptide | Ac-VQIVYK-R₉-NH₂ | 2195 | 2195 | — | — | — |
| V-peptide | Ac-vqvvyk-R₉-NH₂ | 2182 | 2184 | 10 | 3.2 | 4.8 |
| VV-peptide | Ac-vqvvvk-R₉-NH₂ | 2118 | 2119 | NA | NA | — |
| F-peptide | Ac-VQIVFK-R₉-NH₂ | 2179 | 2179 | 5.5 | 3.3 | — |
| K-peptide | Ac-vqivkk-R₉-NH₂ | 2161 | 2160 | NA | NA | — |
| T-FITC- | FITC-(Ahx)₂- | 2737 | 2736 | — | — | — |
| peptide | vqivyk-R₉-NH₂ | | | | | |

^{a}Lower case amino acid codes denote D-amino acids.
^{b}Fitting of kinetic data is described in the experimental section.
NA indicates no aggregation was observed as monitored using ThS fluorescence.
The absence of data indicates that no kinetics were measured. We estimate a ±10% error in fitted kinetic parameters.

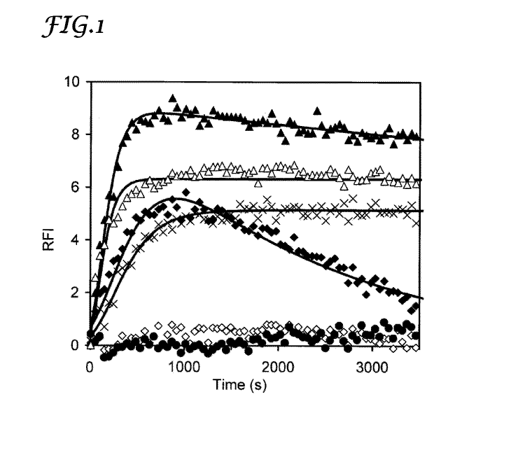

Self-association of peptides in vitro: The inventors measured the self-association behavior of the peptides in vitro in the presence of the polyanions heparin by following the enhancement of fluorescence arising from the binding of ThS to amyloid. The inventors have previously shown that the L-amino acid version of the Core-peptide, Ac-vqivyk-NH₂ (previously referred to AcPHF6), readily polymerizes into amyloid fibrils above a critical concentration of about 2 μM [19, 20]. The polymerization kinetics (shown in FIG. 1) suggest that T-peptide, V-peptide and F-peptide aggregate in a fashion similar to the Core peptide, in spite of their R₉ tag. The inventors fit the kinetic data of these aggregating peptides to growth curves and results are summarized in Table 1. None of the peptides had a significant lag time. The Core-peptide showed the fastest aggregation followed closely by the T-peptide (k=10.6 s⁻¹ and 8.5 s⁻¹, respectively). V-peptide and F-peptide aggregated with about a three times slower rate (3.2-3.3 s⁻¹) while VV-peptide and K-peptide showed no aggregation at the 10 μM concentration. No aggregation was observed for K-peptide or the VV-peptide. In the case of the K-peptide, this finding is consistent with previous findings by the inventors showing that a lysine placed at position 5 in the core sequence resulting in a non-aggregating peptide [19].

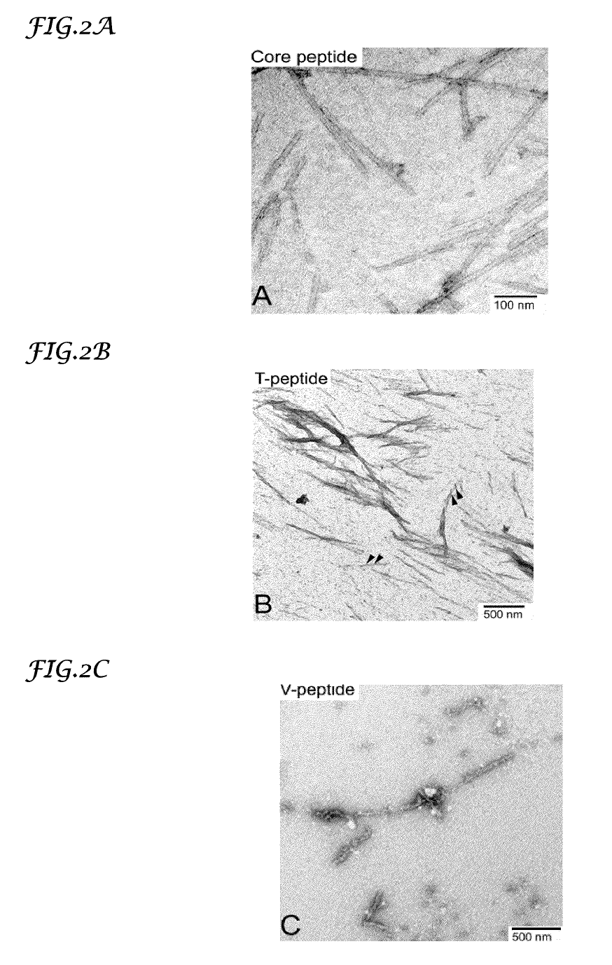

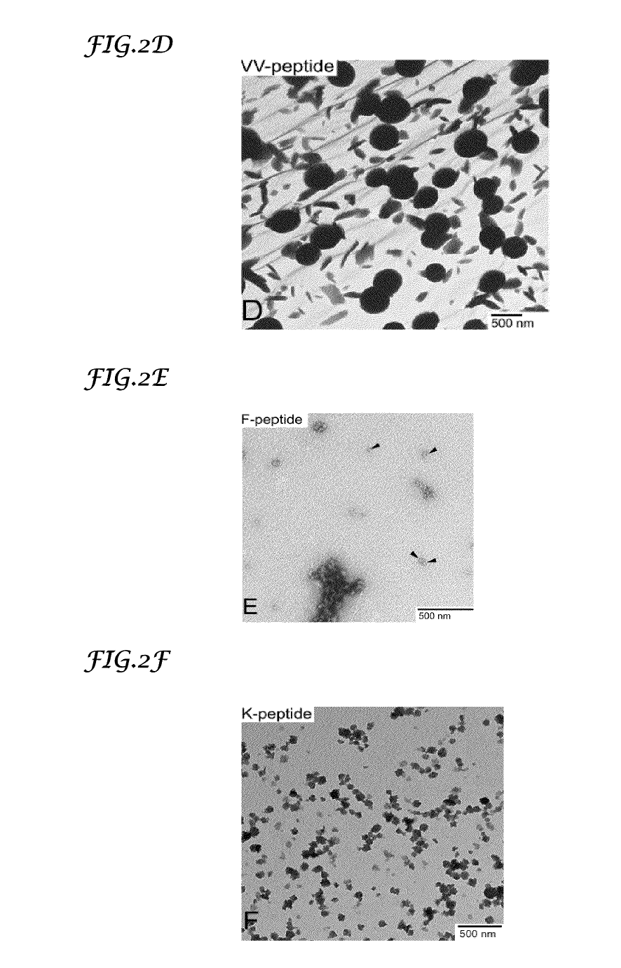

The inventors characterized the morphology of the samples using TEM (FIG. 2A-2F). As previously demonstrated, the Core peptide (2A) formed straight filaments, where each filament is formed from two axially aligned fibrils (7±1 nm diameter) [19]. Small amyloid bodies (20-40 nm diameter) and irregular non-uniform filaments were seen in the TEMs of the V-peptide (2C) while aggregated T-peptide (2B) contained left-handed twisted filaments with a diameter of 27±4 nm and a half-periodicity of 134±12 nm (n=7). Of the peptides not showing significant ThS enhancement over time, irregular shaped non-fibular aggregates appeared in the TEM of K-peptide (2D). The inventors have previously observed such large spherical bodies for other peptides [20].

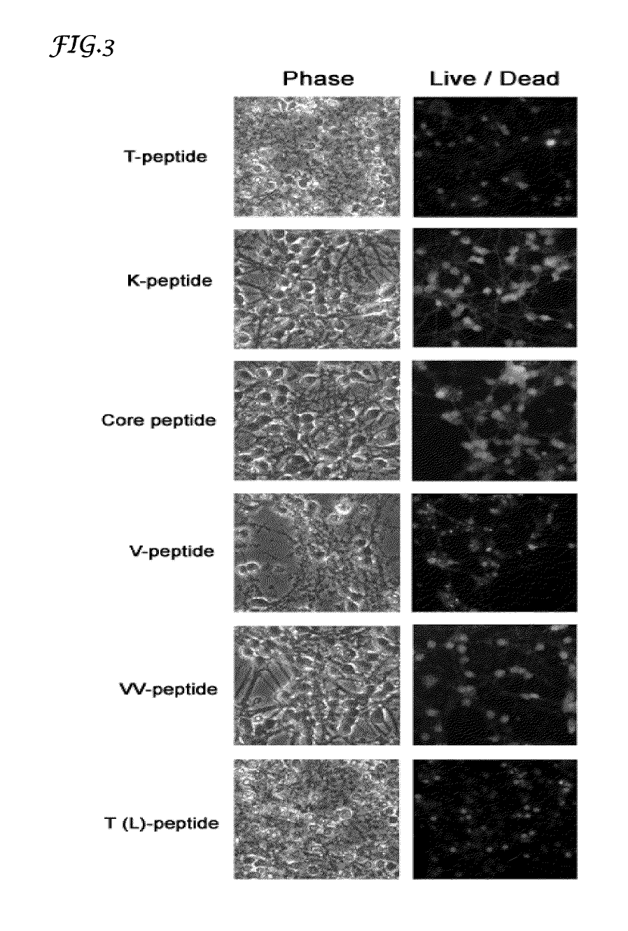

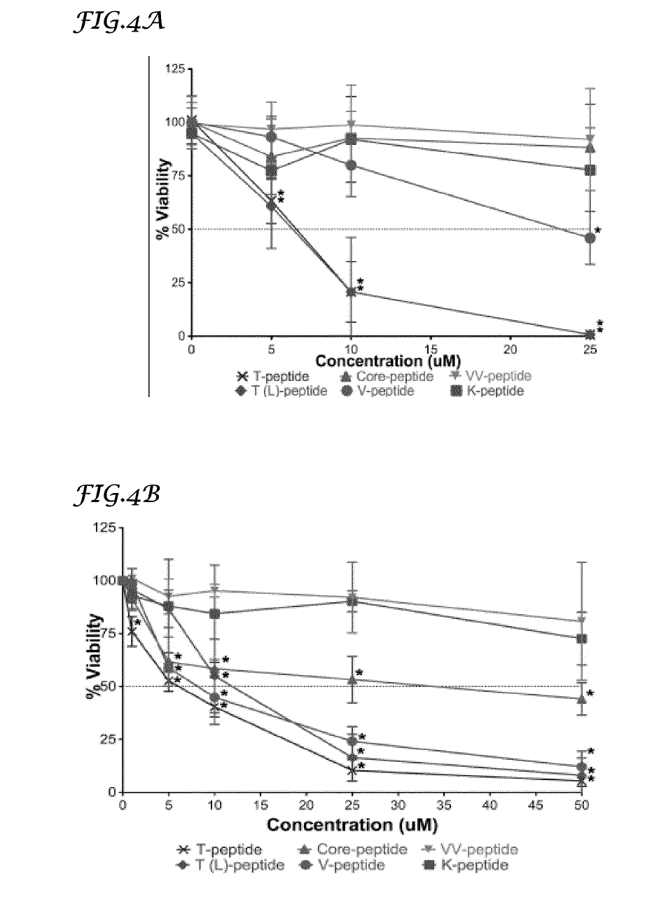

T-peptide is highly neurotoxic: The inventors used cultured cerebellar granule neurons to test the effect of T-peptide exposure on neuronal survival. As shown in FIGS. 3 and 4, T-peptide induces cell death in a concentration-dependent manner with significant death observable at doses of ≧5 μM. About 80% of the neurons were dead when treated with 10 μM of T-peptide. Cell viability results obtained using the Live/Dead assay was confirmed using the MTT assay, which reflects mitochondrial function (FIG. 4B). To rule out the possibility that the R₉ stretch was contributing to the neurotoxic action of T-peptide, the inventors exposed cells to Ac—R₉—NH₂, a peptide composed of just the nine R residues. This peptide had no effect on neuronal survival (data not shown). To confirm that the toxic effect of T-peptide was due to its aggregation ability rather than chirality, the inventors synthesized an L-version of the peptide in which the core as well as the R₉ tag was synthesized using L-amino acids. This peptide, T(L)-peptide displayed a level of neurotoxicity that was similar to T-peptide (FIGS. 3 and 4).

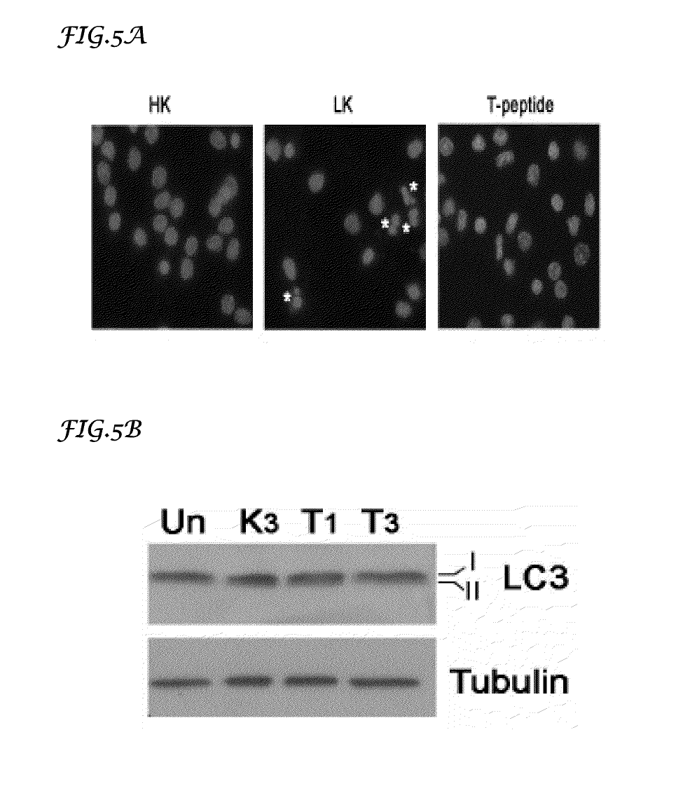

T-peptide treated neurons display disintegration of neurites and cell body shrinkage and fragmentation suggesting that death was due to apoptosis. A characteristic feature of apoptotic death is nuclear condensation and fragmentation. To confirm that apoptosis was the mode by which T-peptide-mediated neuronal death occurs; the inventors visualized nuclei using DAPI. Surprisingly, nuclei of neuronal cultures treated with T-peptide were rarely fragmented and were only slightly condensed (FIG. 5A). In contrast, neurons treated with non-depolarizing medium (low potassium-treatment), a stimulus known to induce apoptosis in cultured cerebellar granule neurons, show robust nuclear condensation and fragmentation (FIG. 5B). Nuclei of T-peptide treated neurons were also abnormally shaped and displayed a highly punctate staining pattern with DAPI (FIG. 5A). These results demonstrated that death induced by T-peptide occurred through a mechanism that was not fully apoptotic.

Another common mode of cell death, and one that occurs in a variety of in vitro and in vivo models of neurodegeneration, is autophagy. Autophagic death is characterized by the appearance of large numbers of vesicles called autophagosomes that are detectable by light microscopy. A reliable biochemical marker of autophagic cell death is the conversion of microtubule-associated protein light chain 3 (LC3) from a cytoplasmic form, referred to as LC-1, to a processed form, LC3-II, which is a component of the autophagosome membrane [30]. Increase in LC3-II levels has been shown to correlate with autophagosome formation. The inventors did not detect the formation of LC3-containing vesicles in T-peptide treated neurons by immunocytochemistry (data not shown). Moreover, LC3-I cleavage to LC3-II was not increased in neurons that were degenerating after treatment with T-peptide (FIG. 4B). These observations suggest that death of neurons resulting from T-peptide exposure is not due to autophagy.

As observed with cerebellar granule neurons, T-peptide induces the death of cultured cortical neurons. However, cortical neurons were slightly less sensitive to T-peptide induced toxicity than cerebellar granule neurons (FIG. 4). As observed with cerebellar granule neurons, no nuclear fragmentation was observed in T-peptide treated cortical neurons (data not shown).

T-peptide neurotoxicity is aggregation-dependent and is enhanced by cell permeability: To examine whether aggregation is necessary for T-peptide induced neurotoxicity, the inventors synthesized three mutant peptides referred to as K-peptide, V-peptide, and VV-peptide (Table 1). TEM and kinetic analyses reveal that neither K-peptide nor VV-peptide aggregate in vitro at concentrations used to measure toxicity. In contrast, V-peptide does form aggregates, albeit less efficiently than T-peptide (Table 1 and FIG. 1). While treatment of cerebellar granule neurons with V-peptide resulted in a moderate level of cell death (˜50% cell death at 25 uM peptide concentration), neither K-peptide nor VV-peptide reduced neuronal survival indicating that neurotoxicity is dependent on peptide aggregation (FIGS. 3 and 4).

Previous studies have shown that tau protein is toxic to neurons even when added extracellularly [18, 31, 32]. To examine whether entry into the cell was required for T-peptide neurotoxicity, the inventors treated cerebellar granule neurons with Core-peptide. Phase contrast microscopy and Live/Dead assays showed that Core-peptide was not toxic (FIGS. 3 and 4A). This demonstrated that the neurotoxic effect of the hexameric tau sequence is mediated intracellularly. Surprisingly, given the healthy appearance of the cultures and results from the Live/Dead assay, MTT assays revealed mitochondrial dysfunction in Core-peptide treated neurons (FIG. 4B).

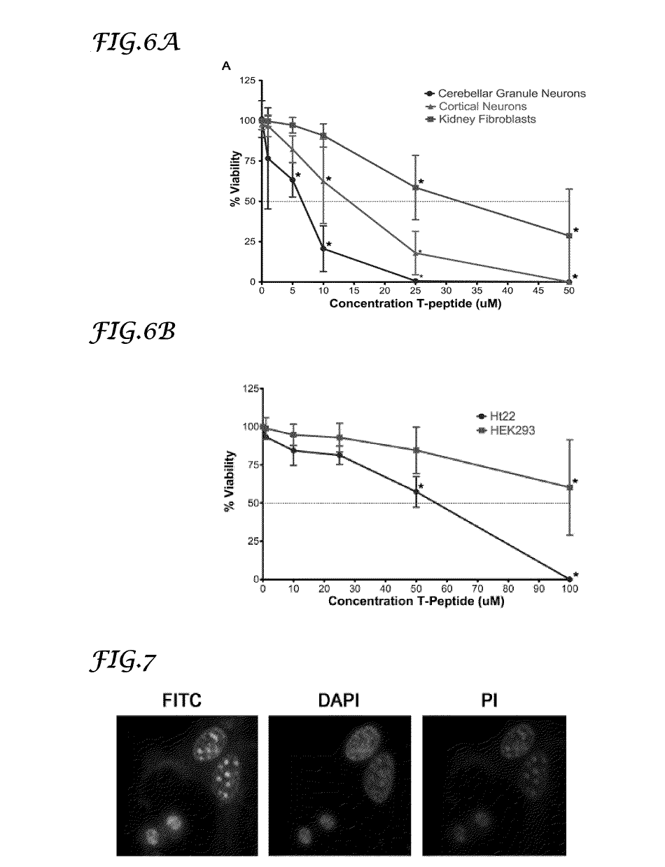

Neurons are selectively sensitive to T-peptide toxicity: In AD and other taupathies, neurons are selectively affected. To examine if T-peptide toxicity is similarly cell selective, the inventors exposed primary cultures of kidney fibroblasts to it. Although these cells were also killed by T-peptide, they were much less sensitive than the two neuronal cell types (FIG. 6A). Primary glial cells are also less sensitive than neurons to T-peptide toxicity (data not shown).

The inventors also treated two cell lines with T-peptide. While the hippocampally-derived HT-22 neuroblastoma cell line shows a moderate level of sensitivity, the human embryonic kidney HEK293T cell line is resistant at doses of up to 50 μM. (FIG. 6B). These results indicate that neuronal cells are more vulnerable to the toxic effect of T-peptide than other cell types.

Peptide aggregates localize to the nuclei within cells: To investigate whether T-peptide enters the cell, the inventors synthesized and treated HT-22 cells with a fluorescent (FITC-conjugated) form of T-peptide. As shown in FIG. 7, T-FITC enters the cell. Interestingly, the peptide localizes predominantly in the nucleus appearing in large and distinct structures resembling aggregates.

To confirm that T-peptide forms aggregates, the inventors used thioflavin-S, a dye which binds protein fibrils and aggregates. As observed with T-FITC, thioflavin-S staining also revealed distinct aggregate-like structures in the nucleus (FIG. 8). In addition to the nucleus, however, a significant level of staining was observed in the cytoplasm. Staining in the cytoplasm was diffuse compared to the punctate pattern in the nucleus. Positive staining with thioflavin-S in the nucleus and cytoplasm was also observed with V-peptide. In contrast, cultures treated with K-peptide were not stained with thioflavin-S.

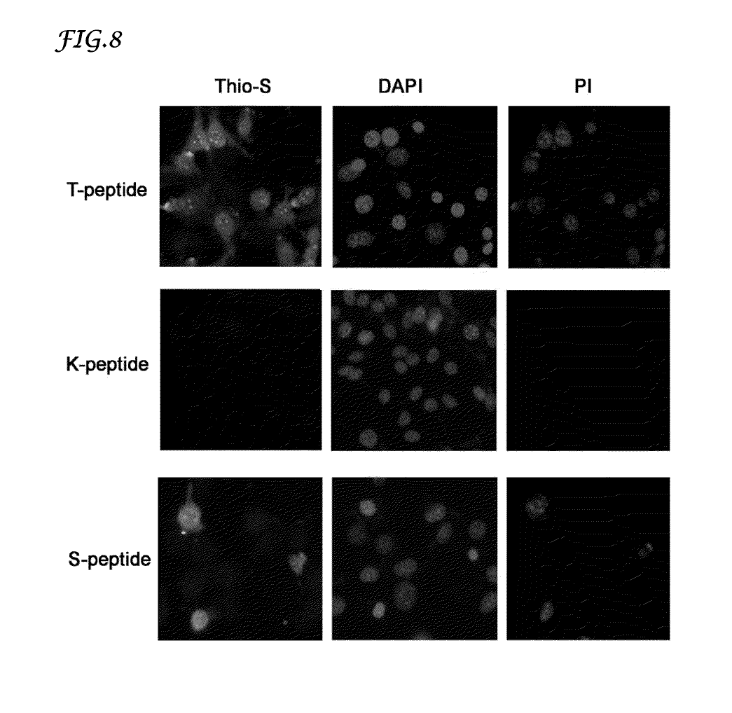

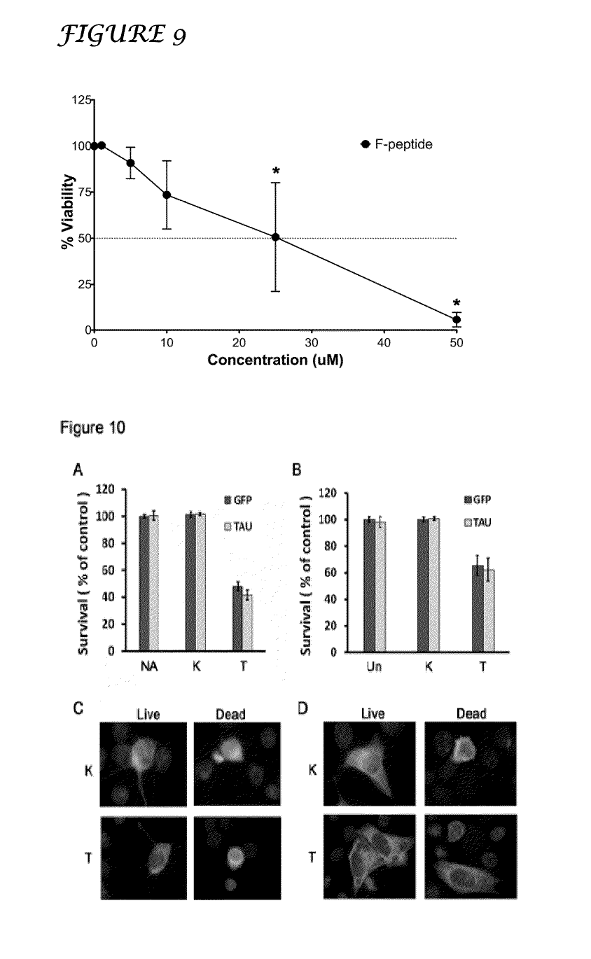

Phosphorylation of T-peptide is not required for aggregation or neurotoxicity: A number of studies have suggested that hyperphosphorylation of Tau within the repeat regions causes its disassociation from microtubules and its assembly into fibrils [33]. Moreover, selective and broad-range kinase inhibitors can reduce abnormal tau phosphorylation and aggregation in transgenic mice affording protection against neuronal degeneration [34, 35]. Recent reports have established that tau is not only phosphorylated on serine and threonine but also on tyrosine residues [36]. The hexameric sequence within T-peptide has a tyrosine residue, which is missing in the two non-toxic peptides, K-peptide and VV-peptide, but is present in the neurotoxic V-peptide. It was conceivable therefore, that phosphorylation of the tyrosine residue within T-peptide was necessary for aggregation and neurotoxicity. To investigate this issue, the inventors synthesized a peptide designated as F-peptide, that has a phenylalanine residue instead of tyrosine. As shown in Table 1, F-peptide is capable of self-aggregation in vitro. As shown in FIG. 9, treatment of neurons with F-peptide induces toxicity. Although the level of toxicity was slightly lower than that displayed by T-peptide, this likely reflects the lower aggregation ability of F-peptide compared with T-peptide. This result demonstrates that phosphorylation of T-peptide is not necessary for neurotoxicity, and strengthens the conclusion that aggregation is the key requirement for neurotoxicity.

Elevated level on endogenous tau does not predispose neurons to T-peptide-induced death. The finding that neurons are selectively sensitive to T-peptide-induced toxicity raises the interesting possibility that the intracellular molecular composition of neurons is responsible for their higher sensitivity. One molecule expressed selectively in neurons that could confer such vulnerability is tau itself In support of such a possibility is the finding that tau-deficient primary neurons are resistant to Aβ-induced toxicity (Liu et al., 2004; Rapoport et al., 2002). Furthermore, ectopic expression of tau fragments in cultured cells and transgenic mice has been reported in some studies, to promote aggregation of endogenous tau (Eckermann et al., 2007; Mocanu et al., 2008a; Wang et al., 2007). To examine the contribution of tau to T-peptide-induced neurotoxicity, we overexpressed human tau (2N-4R isoform) in neurons and then treated the cultures with T or K-peptide. Overexpression of tau had no effect on cell viability by itself. Furthermore, the higher expression did not increase the extent of cell death that resulted from treatment with T-peptide (FIG. 10).

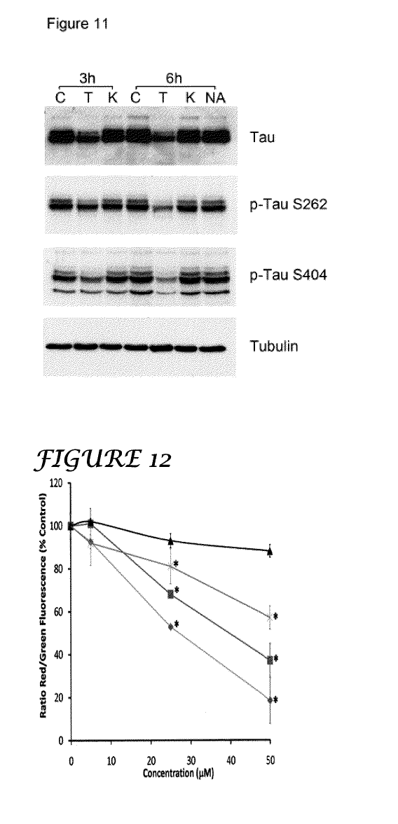

Since T-peptide induces aggregation of cellular proteins and given the proposition that tau fragments including those containing the VQIVYK sequence have been shown to induce tau aggregation both in vivo and in cultured cells (Frost et al., 2009; Mocanu et al., 2008b; Wang et al., 2007), we investigated whether T-peptide causes aggregation of intracellular tau in neurons. As shown in FIG. 11, the overall pattern of ectopically-expressed tau-YPP immunoreactivity was similar in neurons treated with T-peptide or K-peptide. Although the level of immunoreactivity was lower in T-peptide treated neurons, reflecting the reduced level of expression, a similar finding was made in immunocytochemical analysis of endogenous tau (using a tau antibody) in untransfected neurons treated with the two peptides. These results suggest that the aggregates stained with thioflavin-S in T-peptide-treated neurons are not composed of tau.

Treatment with T-peptide results in the downregulation of endogenous tau levels. Although their role in promoting neuronal death remains to be resolved, hyperphosphorylation and aggregation of tau is known occur in AD and other tauopathies. [reviewed in (Castellani et al., 2008)]. We examined whether tau was affected in neurons treated with T-peptide. As shown in FIG. 11, endogenous tau expression was reduced at 3 h of T-peptide with even more reduction occurring at 6 h. In comparison, tubulin expression was not altered. We have also examined the expression of other proteins in separate experiments and have found no change in response to treatment with T-peptide. To examine if phosphorylation of tau was also affected, we used antibodies specific for tau phosphorylated at Ser404 and Ser262. As shown in FIG. 11, the phosphorylation at both of these residues was also reduced. However, the reduction in phosphorylation between T-treated and core or K-treated cultures was similar in extent to that observed with total tau antibody, suggesting that T-peptide does not have a significant effect on the phosphorylation of endogenous tau (FIG. 11).

T-peptide causes a decrease in the mitochondrial membrane potential. Although the MTT assay is commonly used to quantify cell viability, the assays actually evaluates mitochondrial function. Besides confirming loss of viability, our results using the MTT assays raises the possibility that T-peptide affects neuronal viability of cells via modulating the mitochondrial membrane potential. Alteration in mitochondrial membrane potential and mitochondrial dysfunction is a hallmark of neurodegenerative diseases. In order to directly examine the effects of T-peptide on the mitochondrial membrane potential, we used the fluorescent cationic dye, JC-1 (Reers et al., 1991). As shown in FIG. 12, our results indicate that T-peptide caused a reduction in the mitochondrial membrane potential of HT-22 cells in a concentration-dependent manner. Similarly, T (L)-peptide also resulted in a reduction in the mitochondrial membrane potential in a dose-dependent manner. In contrast, there was very little change in the mitochondrial membrane potential when K-peptide was added to HT-22 cells (FIG. 12). Core-peptide also caused a reduction in the mitochondrial membrane potential of HT-22 cells when added at high concentrations, in agreement with our results obtained using MTT assays (FIG. 12).

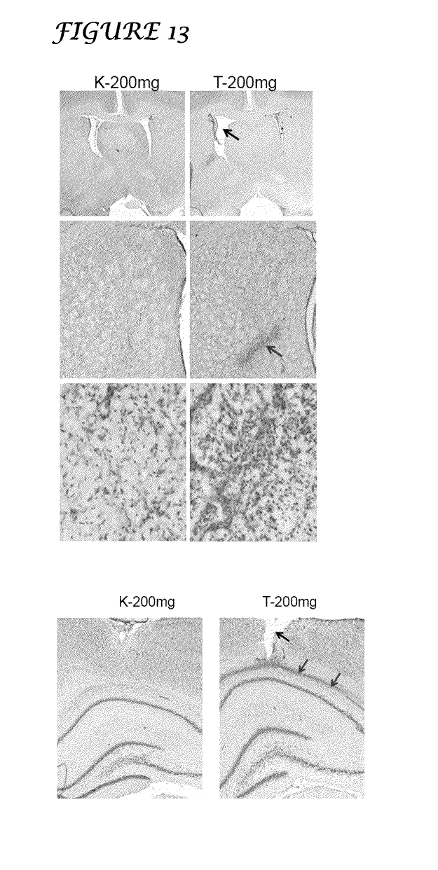

Other aggregating peptides unrelated to tau are not neurotoxic. Although our data indicate that aggregation of T-peptide is required for neurotoxicity, it does not rule out the possibility that any aggregating peptide, regardless of the sequences that it is composed of, would also be toxic. To examine this issue, we synthesized two peptides, STVIIE-R9 (designated at ST peptide), previously described by Pastor et al. (Pastor et al., 2008) and another de novo peptide, SQIVYA-R9. We were able to follow the kinetics of aggregation of both peptides into amyloid forming structures using the fluorescence of thioflavin-S and we characterized their morphology by TEM. The ST-peptide aggregated quite slowly over about a 3000 s lag time after which it showed a second period of more rapid aggregation kinetics (A=4.9, k=1.6×10-3 s-1; A′=6.3, k′=5.1×10-3 s-1 according to Eqn. [2]), while SQ-peptide showed kinetics similar to V-peptide (A=8.8, k=3.8×10-3 s-1). The ST-peptide formed amyloid sheets which appear to roll into irregular fibers 10-40 nm in diameter while the SQ-peptide formed irregular non-uniform filaments similar to V-peptide (FIG. 2). As shown in FIG. 13, neither of these two peptides displayed neurotoxicity when tested in culture. This suggests that in addition to its ability to aggregate, the neurotoxic action of T-peptide is dependent on its sequence. Peptide induces neurodegeneration in the intact rat brain. While exposure to cultured neurons to T-peptide resulted in cell death, the effects of this peptide in vivo remained unclear. To examine this issue, 200 mg of T-peptide and K-peptide was sterotaxically injected into adult rat brains. Each animal received 2 injections—on one side into the striatum, and on the other side into the hippocampus. Rats were analyzed 1 week later. T-peptide-injected rats do display more damage than K-peptide-injected rats. While the hippocampus does not seem to show much damage, the corpus callosum shows damage and scarring. The striatum shows obvious damage and the lateral ventricle is enlarged consistent with cell atrophy and/or loss. As seen in FIG. 13 the black arrow shows enlarged ventricle; Red arrow shows damaged area within the Lowest panels show high mag view of middle panel. In the lower image of FIG. 13 the black arrow shows more degeneration in cortex Red arrows show scarring of the corpus callosum presumably after damage. No damage is seen in the hippocampus however.

The present invention describes the establishment of a convenient cell culture model to study the molecular mechanisms underlying tau neurotoxicity using an exogenously provided synthetic peptide containing the sequence, VQIVYK, located in the R3 region of tau. This hexameric sequence is known to self-assemble into fibrils robustly and with rapid kinetics. The presence of this hexameric motif within full-length tau and tau fragments is necessary their oligomerization into a β-sheet conformation (Sahara et al., 2007). Moreover, this sequence is part of the protease-resistant PHF core identified in AD brains, and of tau fragments found in AD and in experimental models of AD (Gamblin et al., 2003)

How does this model relate to the neurodegeneration observed in AD? In AD, aggregated tau deposits first appear in the entorhinal cortex spreading to the hippocampus and eventually to most of the cortex. The gradual spreading of tau aggregates and neurodegeneration raises the possibility that healthy neurons and brain regions are exposed to some exogenous toxic factor released from degenerating neurons. It has been proposed that extracellular tau and proteolytic fragments of it could itself represent the toxic factor (Frost et al., 2009; Gomez-Ramos et al., 2006; Hernandez and Avila, 2008). Besides being released from dying neurons, tau fragments could leech out from tau “ghost tangles”. It is likely that toxic tau fragments contain within them sequences such the T-peptide core sequence, which are capable of initiating fibrillization of the fragment. Although we have facilitated cell entry by addition of the R₉ tag in our model system, it is possible that in disease states tau fragments are taken up by neurons, albeit inefficiently, by other mechanisms. Cytotoxicity by extracellularly-located aggregating peptides has been described by other investigators, and mechanisms such as interaction of aggregated peptide to lipids of the membrane, formation of membrane pores, and alterations in membrane properties such as conductance or fluidity have been suggested (Kayed et al., 2004; Quist et al., 2005; Rochet et al., 2004; Singer and Dewji, 2006). In our study, Core-peptide, which lacks the cell-permeabilizing R₉ tag, is not significantly neurotoxic. These analyses were done at 24 h after peptide treatment. It is possible that long-term treatment with Core-peptide would elicit some neurotoxicity as others have reported with full-length or fragments of tau.

Whether tau aggregation is protective or harmful has been an unresolved issue. Our results indicate that aggregation of tau is necessary for neurotoxicity. Two separate mutant forms of the T-peptide that are incapable of assembling into filaments (K-peptide and VV-peptide) are not toxic, whereas two other mutant forms of the peptide that are capable of aggregating (V-peptide and F-peptide), are toxic. These results are consistent with previously published studies in which tau toxicity was found to be higher in cell lines transfected with an aggregation-prone mutant form of tau, while cells expressing aggregation-deficient mutants showed reduced toxicity (Khlistunova et al., 2006; Khlistunova et al., 2007). Also consistent with an essential role for aggregation is the finding that pharmacological antagonists of aggregation are protective in mammalian cell culture and Drosophila models of tau neurotoxicity (Berger et al., 2006; Khlistunova et al., 2006; Khlistunova et al., 2007). Interestingly, other hexameric peptides with sequences unrelated to T-peptide or tau but capable of aggregating efficiently in vitro do not display neurotoxicity. This suggests that while aggregation is necessary for neurotoxicity, degeneration induced by T-peptide is dependent on its sequence. Our previous studies with peptides of the sequence VQIVXK have shown that even single amino acid substitutions can dramatically affect their ability to form β-sheet structure in solution and the fraction of peptide existing as insoluble amyloid (Rojas Quijano et al., 2006). Circular dichroism spectra of the R9 tagged peptides (data not shown) suggest that the greatest fraction of the peptide present at equilibrium exists as random coil and fails to highlight any conformational differences between different peptides.

The effect of T-peptide on cell viability was studied on several different types of primary cells and cell lines. These included cultures of cerebellar granule neurons, cortical neurons, cortical glial cultures and kidney fibroblasts, as well as HT-22 neuroblastoma and the embryonic kidney HEK293T cell lines. We find that primary neurons are most sensitive to T-peptide induced neurotoxicity. HT-22 cells were also moderately sensitive to T-peptide and showed a higher level of toxicity than either kidney fibroblasts or HEK293 cells. Although relative uptake of peptide or its stability has not been analyzed in the different cell types, this finding raises the interesting possibility that the intracellular molecular composition of neurons is responsible for their higher sensitivity to the toxic effects of aggregated T-peptide. Another effect of T-peptide induced neurotoxicity that recapitulates AD and other neurodegenerative diseases is that it induces mitochondrial dysfunction as indicated by reduced membrane potential and lowered ability to reduce MTT to formazan. Intriguingly, mitochondrial dysfunction is also induced by Core-peptide, although this peptide does not cause cell death. This raises the possibility that mitochondrial dysfunction by itself may not be sufficient to induce cell death. Other biochemical alterations induced by T-peptide that require entry into the cell, such as downregulation of tau levels, could be essential for neuronal death.

The utilization of a fluorescently-labeled form of T-peptide has permitted the determination of its sub-cellular localization. Although some staining is visible in the cytoplasm, T-peptide localizes predominantly to the nucleus. Interestingly, within the nucleus T-peptide is in large aggregates. It is not clear whether the nuclear aggregates are composed solely of T-peptide, or whether other proteins are also contained within them. While thioflavin-S staining confirmed the presence of nuclear aggregates, more staining with this dye was observed in the cytoplasm where staining with fluorescent T-peptide is weak, at best. These results suggest that the cytoplasmic aggregates, the formation of which is induced by T-peptide, are composed primarily of endogenous proteins. One candidate endogenous protein that could be induced to aggregate in response to T-peptide is tau. Previous studies have suggested that the fragments containing the hexameric sequence within T-peptide are capable of nucleating fibrillization of full-length tau in cultured cells as well as transgenic mice (Frost et al., 2009; Mocanu et al., 2008b; Wang et al., 2007). To examine this possibility, we examined the effect of T-peptide on the localization of endogenous tau and transfected tau-YPF. Interestingly, T-peptide had no effect on the distribution or appearance of intracellular tau arguing against the possibility that neurotoxicity of T-peptide was mediated through the aggregation of endogenous tau. While not affecting localization, treatment with T-peptide does result in a dramatic reduction in tau levels. Fractionation of soluble and insoluble tau from T-peptide-treated neurons showed a distribution similar to neurons treated with K-peptide (data not shown) ruling out the possibility that the reduction is due to the conversion of tau into an insoluble form through aggregation. Another possibility is that T-peptide induces the proteolytic cleavage of tau. Such cleavage of tau is known to occur in AD and in other experimental models of neurodegeneration (Canu et al., 1998; Corsetti et al., 2008; Gamblin et al., 2003).

The present invention provides a tissue culture model that recapitulates features of the selective degeneration of neurons seen in AD. Although it is unclear whether the hexameric peptide itself is present in AD brain or cerebrospinal fluid, it is within the 12 kDa tau protein protease-resistant minimal component of PHFs isolated from AD brains Oakes et al., 1991; Novak et al., 1993; Wischik et al., 1988a; Wischik et al., 1988b). It is also part of tau fragments produced in AD brains and experimental models that are capable of promoting neurodegeneration when expressed in experimental systems (Gamblin et al., 2003). Once released by dying neurons, such tau fragments can be taken up by healthy neurons by an endocytotic mechanism where they cause a reduction in endogenous tau levels, possibly through the induction of proteolytic cleavage. Mitochondrial dysfunction, another characteristic feature of AD is also observed following exposure to T-peptide. The tissue culture model we have described can be used to study the molecular mechanisms underlying tau neurotoxicity. Moreover, it is amenable for high-throughput screening of chemical libraries for blockers of tau fragment fibrillization or its cytotoxic effect on neurons and neuronal cell lines. Our results also suggest that an immunotherapeutic approach specifically targeting the hexameric sequence within T-peptide might reduce neurodegeneration in AD and other taupathies. Although the present invention is discussed in the example of T-peptide in cell culture—others could neurotoxicity systems may be used including yeast, flies, worms, and transgenic mice or administering mice/rats with it. For example, FIG. 13 shows administration of the peptide in the brains of rats produces neurotoxicity.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 6,479,528: Methods for inhibiting or reversing tau filament formation polymerization.

U.S. Pat. No. 6,803,233: Model for Alzheimer's disease and other neurodegenerative diseases.

European Patent No. 1373529 B1: Tau-opathy model.

United States Patent Publication No. 20090162336: Novel Tools for the Diagnosis and Treatment of Alzheimer's Disease.

[1] Feany, M. B. and Dickson, D. W. (1996) Neurodegenerative disorders with extensive tau pathology: A comparative study and review. Ann. Neurol. 40, 139-148.

[2] Ballatore, C., Lee, V. M., Trojanowski, J. Q. (2007) Tau-mediated neurodegeneration in alzheimer's disease and related disorders. Nat. Rev. Neurosci. 8, 663-672.

[3] Honson, N. S. and Kuret, J. (2008) Tau aggregation and toxicity in tauopathic neurodegenerative diseases. J. Alzheimers Dis. 14, 417-422.

[4] Castellani, R. J., Nunomura, A., Lee, H. G., Perry, G., Smith, M. A. (2008) Phosphorylated tau: Toxic, protective, or none of the above. J. Alzheimers Dis. 14, 377-383.

[5] Canu, N., Dus, L., Barbato, C., Ciotti, M. T., Brancolini, C., Rinaldi, A. M., Novak, M., Cattaneo, A., Bradbury, A., Calissano, P. (1998) Tau cleavage and dephosphorylation in cerebellar granule neurons undergoing apoptosis. J. Neurosci. 18, 7061-7074.

[6] Corsetti, V., Amadoro, G., Gentile, A., Capsoni, S., Ciotti, M. T., Cencioni, M. T., Atlante, A., Canu, N., Rohn, T. T., Cattaneo, A., Calissano, P. (2008) Identification of a caspase-derived N-terminal tau fragment in cellular and animal alzheimer's disease models. Mol. Cell. Neurosci. 38, 381-392.

[7] Gamblin, T. C., Chen, F., Zambrano, A., Abraha, A., Lagalwar, S., Guillozet, A. L., Lu, M., Fu, Y., Garcia-Sierra, F., LaPointe, N., Miller, R., Berry, R. W., Binder, L. I., Cryns, V. L. (2003) Caspase cleavage of tau: Linking amyloid and neurofibrillary tangles in alzheimer's disease. Proc. Natl. Acad. Sci. U.S.A. 100, 10032-10037.

[8] Schneider, A., Biernat, J., von Bergen, M., Mandelkow, E., Mandelkow, E. M. (1999) Phosphorylation that detaches tau protein from microtubules (Ser262, Ser214) also protects it against aggregation into alzheimer paired helical filaments. Biochemistry. 38, 3549-3558.

[9] Wang, Y. P., Biernat, J., Pickhardt, M., Mandelkow, E., Mandelkow, E. M. (2007) Stepwise proteolysis liberates tau fragments that nucleate the alzheimer-like aggregation of full-length tau in a neuronal cell model. Proc. Natl. Acad. Sci. U.S.A. 104, 10252-10257.

[10] Andorfer, C., Acker, C. M., Kress, Y., Hof, P. R., Duff, K., Davies, P. (2005) Cell-cycle reentry and cell death in transgenic mice expressing nonmutant human tau isoforms. J. Neurosci. 25, 5446-5454.

[11] Alonso Adel, C., Li, B., Grundke-Iqbal, I., Iqbal, K. (2006) Polymerization of hyperphosphorylated tau into filaments eliminates its inhibitory activity. Proc. Natl. Acad. Sci. U. S. A. 103, 8864-8869.

[12] Berger, Z., Roder, H., Hanna, A., Carlson, A., Rangachari, V., Yue, M., Wszolek, Z., Ashe, K., Knight, J., Dickson, D., Andorfer, C., Rosenberry, T. L., Lewis, J., Hutton, M., Janus, C. (2007) Accumulation of pathological tau species and memory loss in a conditional model of tauopathy. J. Neurosci. 27, 3650-3662.

[13] Ding, H. and Johnson, G. V. (2008) The last tangle of tau. J. Alzheimers Dis. 14, 441-447.

[14] Santacruz, K., Lewis, J., Spires, T., Paulson, J., Kotilinek, L., Ingelsson, M., Guimaraes, A., DeTure, M., Ramsden, M., McGowan, E., Forster, C., Yue, M., Orne, J., Janus, C., Mariash, A., Kuskowski, M., Hyman, B., Hutton, M., Ashe, K. H. (2005) Tau suppression in a neurodegenerative mouse model improves memory function. Science. 309, 476-481.

[15] von Bergen, M., Friedhoff, P., Biernat, J., Heberle, J., Mandelkow, E. M., Mandelkow, E. (2000) Assembly of tau protein into alzheimer paired helical filaments depends on a local sequence motif ((306)VQIVYK(311)) forming beta structure. Proc. Natl. Acad. Sci. U.S.A. 97, 5129-5134.

[16] von Bergen, M., Barghorn, S., Li, L., Marx, A., Biernat, J., Mandelkow, E. M., Mandelkow, E. (2001) Mutations of tau protein in frontotemporal dementia promote aggregation of paired helical filaments by enhancing local beta-structure. J. Biol. Chem. 276, 48165-48174.

[17] Mocanu, M. M., Nissen, A., Eckermann, K., Khlistunova, I., Biernat, J., Drexler, D., Petrova, O., Schonig, K., Bujard, H., Mandelkow, E., Zhou, L., Rune, G., Mandelkow, E. M. (2008) The potential for beta-structure in the repeat domain of tau protein determines aggregation, synaptic decay, neuronal loss, and coassembly with endogenous tau in inducible mouse models of tauopathy. J. Neurosci. 28, 737-748.

[18] Frost, B., Jacks, R. L., Diamond, M. I. (2009) Propagation of tau misfolding from the outside to the inside of a cell. J. Biol. Chem. 284, 12845-12852.

[19] Rojas Quijano, F. A., Morrow, D., Wise, B. M., Brancia, F. L., Goux, W. J. (2006) Prediction of nucleating sequences from amyloidogenic propensities of tau-related peptides. Biochemistry. 45, 4638-4652.

[20] Goux, W. J., Kopplin, L., Nguyen, A. D., Leak, K., Rutkofsky, M., Shanmuganandam, V. D., Sharma, D., Inouye, H., Kirschner, D. A. (2004) The formation of straight and twisted filaments from short tau peptides. J. Biol. Chem. 279, 26868-26875.

[21] Sahara, N., Maeda, S., Murayama, M., Suzuki, T., Dohmae, N., Yen, S. H., Takashima, A. (2007) Assembly of two distinct dimers and higher-order oligomers from full-length tau. Eur. J. Neurosci. 25, 3020-3029.

[22] D'Mello, S. R., Galli, C., Ciotti, T., Calissano, P. (1993) Induction of apoptosis in cerebellar granule neurons by low potassium: Inhibition of death by insulin-like growth factor I and cAMP. Proc. Natl. Acad. Sci. U.S.A. 90, 10989-10993.

[23] Koulich, E., Nguyen, T., Johnson, K., Giardina, C., D'mello, S. (2001) NF-kappaB is involved in the survival of cerebellar granule neurons: Association of IkappaBbeta [correction of ikappabeta] phosphorylation with cell survival. J. Neurochem. 76, 1188-1198.

[24] Barghorn, S. and Mandelkow, E. (2002) Toward a unified scheme for the aggregation of tau into alzheimer paired helical filaments. Biochemistry. 41, 14885-14896.

[25] von Bergen, M., Barghorn, S., Muller, S. A., Pickhardt, M., Biernat, J., Mandelkow, E. M., Davies, P., Aebi, U., Mandelkow, E. (2006) The core of tau-paired helical filaments studied by scanning transmission electron microscopy and limited proteolysis. Biochemistry. 45, 6446-6457.

[26] Futaki, S. (2002) Arginine-rich peptides: Potential for intracellular delivery of macromolecules and the mystery of the translocation mechanisms. Int. J. Pharm. 245, 1-7.

[27] Futaki, S., Suzuki, T., Ohashi, W., Yagami, T., Tanaka, S., Ueda, K., Sugiura, Y. (2001) Arginine-rich peptides. an abundant source of membrane-permeable peptides having potential as carriers for intracellular protein delivery. J. Biol. Chem. 276, 5836-5840.

[28] Allen, M. J., MacRenaris, K. W., Venkatasubramanian, P. N., Meade, T. J. (2004) Cellular delivery of MRI contrast agents. Chem. Biol. 11, 301-307.

[29] Suzuki, T., Futaki, S., Niwa, M., Tanaka, S., Ueda, K., Sugiura, Y. (2002) Possible existence of common internalization mechanisms among arginine-rich peptides. J. Biol. Chem. 277, 2437-2443.

[30] Kabeya, Y., Mizushima, N., Ueno, T., Yamamoto, A., Kirisako, T., Noda, T., Kominami, E., Ohsumi, Y., Yoshimori, T. (2000) LC3, a mammalian homologue of yeast Apg8p, is localized in autophagosome membranes after processing. EMBO J. 19, 5720-5728.

[31] Gomez-Ramos, A., Diaz-Hernandez, M., Cuadros, R., Hernandez, F., Avila, J. (2006) Extracellular tau is toxic to neuronal cells. FEBS Lett. 580, 4842-4850.

[32] Gomez-Ramos, A., Diaz-Hernandez, M., Rubio, A., Miras-Portugal, M. T., Avila, J. (2008) Extracellular tau promotes intracellular calcium increase through M1 and M3 muscarinic receptors in neuronal cells. Mol. Cell. Neurosci. 37, 673-681.

[33] Takashima, A. (2008) Hyperphosphorylated tau is a cause of neuronal dysfunction in tauopathy. J. Alzheimers Dis. 14, 371-375.

[34] Le Corre, S., Klafki, H. W., Plesnila, N., Hubinger, G., Obermeier, A., Sahagun, H., Monse, B., Seneci, P., Lewis, J., Eriksen, J., Zehr, C., Yue, M., McGowan, E., Dickson, D. W., Hutton, M., Roder, H. M. (2006) An inhibitor of tau hyperphosphorylation prevents severe motor impairments in tau transgenic mice. Proc. Natl. Acad. Sci. U.S.A. 103, 9673-9678.

[35] Noble, W., Planel, E., Zehr, C., Olm, V., Meyerson, J., Suleman, F., Gaynor, K., Wang, L., LaFrancois, J., Feinstein, B., Burns, M., Krishnamurthy, P., Wen, Y., Bhat, R., Lewis, J., Dickson, D., Duff, K. (2005) Inhibition of glycogen synthase kinase-3 by lithium correlates with reduced tauopathy and degeneration in vivo. Proc. Natl. Acad. Sci. U.S.A. 102, 6990-6995.

[36] Lebouvier, T., Scales, T. M., Williamson, R., Noble, W., Duyckaerts, C., Hanger, D. P., Reynolds, C. H., Anderton, B. H., Derkinderen, P. (2009) The microtubule-associated protein tau is also phosphorylated on tyrosine. J. Alzheimers Dis.

[37] Hernandez, F. and Avila, J. (2008) Tau aggregates and tau pathology. J. Alzheimers Dis. 14, 449-452.

[38] Singer, S. J. and Dewji, N. N. (2006) Evidence that perutz's double-beta-stranded subunit structure for beta-amyloids also applies to their channel-forming structures in membranes. Proc. Natl. Acad. Sci. U.S.A. 103, 1546-1550.

[39] Rochet, J. C., Outeiro, T. F., Conway, K. A., Ding, T. T., Volles, M. J., Lashuel, H. A., Bieganski, R. M., Lindquist, S. L., Lansbury, P. T. (2004) Interactions among alpha-synuclein, dopamine, and biomembranes: Some clues for understanding neurodegeneration in parkinson's disease. J. Mol. Neurosci. 23, 23-34.

[40] Quist, A., Doudevski, I., Lin, H., Azimova, R., Ng, D., Frangione, B., Kagan, B., Ghiso, J., Lal, R. (2005) Amyloid ion channels: A common structural link for protein-misfolding disease. Proc. Natl. Acad. Sci. U.S.A. 102, 10427-10432.

[41] Kayed, R., Sokolov, Y., Edmonds, B., McIntire, T. M., Milton, S. C., Hall, J. E., Glabe, C. G. (2004) Permeabilization of lipid bilayers is a common conformation-dependent activity of soluble amyloid oligomers in protein misfolding diseases. J. Biol. Chem. 279, 46363-46366.

[42] Khlistunova, I., Biernat, J., Wang, Y., Pickhardt, M., von Bergen, M., Gazova, Z., Mandelkow, E., Mandelkow, E. M. (2006) Inducible expression of tau repeat domain in cell models of tauopathy: Aggregation is toxic to cells but can be reversed by inhibitor drugs. J. Biol. Chem. 281, 1205-1214.

[43] Khlistunova, I., Pickhardt, M., Biernat, J., Wang, Y., Mandelkow, E. M., Mandelkow, E. (2007) Inhibition of tau aggregation in cell models of tauopathy. Curr. Alzheimer Res. 4, 544-546.

[44] Berger, Z., Ravikumar, B., Menzies, F. M., Oroz, L. G., Underwood, B. R., Pangalos, M. N., Schmitt, I., Wullner, U., Evert, B. O., O'Kane, C. J., Rubinsztein, D. C. (2006) Rapamycin alleviates toxicity of different aggregate-prone proteins. Hum. Mol. Genet. 15, 433-442.

[45] Eckermann, K., Mocanu, M. M., Khlistunova, I., Biernat, J., Nissen, A., Hofmann, A., Schonig, K., Bujard, H., Haemisch, A., Mandelkow, E., Zhou, L., Rune, G., Mandelkow, E. M. (2007) The beta-propensity of tau determines aggregation and synaptic loss in inducible mouse models of tauopathy. J. Biol. Chem. 282, 31755-31765.

[46] Mocanu, M. M., Nissen, A., Eckermann, K., Khlistunova, I., Biernat, J., Drexler, D., Petrova, O., Schonig, K., Bujard, H., Mandelkow, E., Zhou, L., Rune, G., Mandelkow, E. M. (2008) The potential for beta-structure in the repeat domain of tau protein determines aggregation, synaptic decay, neuronal loss, and coassembly with endogenous tau in inducible mouse models of tauopathy. J. Neurosci. 28, 737-748.

[47] Khatoon, S., Grundke-Iqbal, I., Iqbal, K. (1992) Brain levels of microtubule-associated protein tau are elevated in alzheimer's disease: A radioimmuno-slot-blot assay for nanograms of the protein. J. Neurochem. 59, 750-753.

[48] Khatoon, S., Grundke-Iqbal, I., Iqbal, K. (1994) Levels of normal and abnormally phosphorylated tau in different cellular and regional compartments of alzheimer disease and control brains. FEBS Lett. 351, 80-84.

[49] Roberson, E. D., Scearce-Levie, K., Palop, J. J., Yan, F., Cheng, I. H., Wu, T., Gerstein, H., Yu, G. Q., Mucke, L. (2007) Reducing endogenous tau ameliorates amyloid beta-induced deficits in an alzheimer's disease mouse model. Science. 316, 750-754.

[50] Liu, T., Perry, G., Chan, H. W., Verdile, G., Martins, R. N., Smith, M. A., Atwood, C. S. (2004) Amyloid-beta-induced toxicity of primary neurons is dependent upon differentiation-associated increases in tau and cyclin-dependent kinase 5 expression. J. Neurochem. 88, 554-563.

[51] Rapoport, M., Dawson, H. N., Binder, L. I., Vitek, M. P., Ferreira, A. (2002) Tau is essential to beta-amyloid-induced neurotoxicity. Proc. Natl. Acad. Sci. U.S.A. 99, 6364-6369.

[52] Thurston, V. C., Pena, P., Pestell, R., Binder, L. I. (1997) Nucleolar localization of the microtubule-associated protein tau in neuroblastomas using sense and anti-sense transfection strategies. Cell Motil. Cytoskeleton. 38, 100-110.

[53] Thurston, V. C., Zinkowski, R. P., Binder, L. I. (1996) Tau as a nucleolar protein in human nonneural cells in vitro and in vivo. Chromosoma. 105, 20-30.

[54] Greenwood, J. A. and Johnson, G. V. (1995) Localization and in situ phosphorylation state of nuclear tau. Exp. Cell Res. 220, 332-337.

[55] Wang, Y., Loomis, P. A., Zinkowski, R. P., Binder, L. I. (1993) A novel tau transcript in cultured human neuroblastoma cells expressing nuclear tau. J. Cell Biol. 121, 257-267.

[56] Wei, Y., Qu, M. H., Wang, X. S., Chen, L., Wang, D. L., Liu, Y., Hua, Q., He, R. Q. (2008) Binding to the minor groove of the double-strand, tau protein prevents DNA from damage by peroxidation. PLoS One. 3, e2600.

[57] Papasozomenos, S. C. (1995) Nuclear tau immunoreactivity in presenile dementia with motor neuron disease: A case report. Clin. Neuropathol. 14, 100-104.

[58] Sjoberg, M. K., Shestakova, E., Mansuroglu, Z., Maccioni, R. B., Bonnefoy, E. (2006) Tau protein binds to pericentromeric DNA: A putative role for nuclear tau in nucleolar organization. J. Cell. Sci. 119, 2025-2034.

[59] Tsukane, M., Yoshizaki, C., Yamauchi, T. (2007) Development and specific induction of apoptosis of cultured cell models overexpressing human tau during neural differentiation: Implication in alzheimer's disease. Anal. Biochem. 360, 114-122.

[60] Wischik, C. M., Novak, M., Thogersen, H. C., Edwards, P. C., Runswick, M. J., Jakes, R., Walker, J. E., Milstein, C., Roth, M., Klug, A. (1988) Isolation of a fragment of tau derived from the core of the paired helical filament of alzheimer disease. Proc. Natl. Acad. Sci. U.S.A. 85, 4506-4510.

[61] Wischik, C. M., Novak, M., Edwards, P. C., Klug, A., Tichelaar, W., Crowther, R. A. (1988) Structural characterization of the core of the paired helical filament of alzheimer disease. Proc. Natl. Acad. Sci. U.S.A. 85, 4884-4888.

[62] Novak, M., Kabat, J., Wischik, C. M. (1993) Molecular characterization of the minimal protease resistant tau unit of the alzheimer's disease paired helical filament. EMBO J. 12, 365-370.

[63] Jakes, R., Novak, M., Davison, M., Wischik, C. M. (1991) Identification of 3- and 4-repeat tau isoforms within the PHF in alzheimer's disease. EMBO J. 10, 2725-2729.

[64] Goedert, M. and Jakes, R. (1990) Expression of separate isoforms of human tau protein: Correlation with the tau pattern in brain and effects on tubulin polymerization. EMBO J. 9, 4225-4230.

[65] Kanai, Y., Takemura, R., Oshima, T., Mori, H., Ihara, Y., Yanagisawa, M., Masaki, T., Hirokawa, N. (1989) Expression of multiple tau isoforms and microtubule bundle formation in fibroblasts transfected with a single tau cDNA. J. Cell Biol. 109, 1173-1184.

[66] Ding, H. and Johnson, G. V. (2008) The last tangle of tau. J. Alzheimers Dis. 14, 441-447.

[67] Amadoro, G., Serafino, A. L., Barbato, C., Ciotti, M. T., Sacco, A., Calissano, P., Canu, N. (2004) Role of N-terminal tau domain integrity on the survival of cerebellar granule neurons. Cell Death Differ. 11, 217-230.

[68] Bandyopadhyay, B., Li, G., Yin, H., Kuret, J. (2007) Tau aggregation and toxicity in a cell culture model of tauopathy. J. Biol. Chem. 282, 16454-16464.

[69] Bondareff, W., Matsuyama, S. S., Dell'Albani, P. (1998) Production of paired helical filament, tau-like proteins by PC12 cells: A model of neurofibrillary degeneration. J. Neurosci. Res. 52, 498-504.

[70] Frost, B., Jacks, R. L., Diamond, M. I. (2009) Propagation of tau misfolding from the outside to the inside of a cell. J. Biol. Chem. 284, 12845-12852.

[71] Sturzu, A, Klose, U., Echner, H., Beck, A., Gharabaghi, A., Kalbacher, H., Heckl, S. (2009) Amino Acids 29, 249-255.

What is claimed is:

1. An in vitro cell culture model or system for studying one or more characteristics that are indicative of one or more taupathies or neurodegenerative conditions in a subject comprising:
one or more cells or cell-cultures dispersed in a medium, wherein the one or more cells comprise cerebellar granule neurons, glial cells, neuronal cultures, neurons, kidney fibroblasts, hippocampal cells, human embryonic kidney cells, or any combinations thereof;
one or more positive control agents, wherein the positive control agents induce the one or more characteristics that are indicative of one or more taupathies or neurodegenerative conditions in a subjects;
one or more negative control agents, wherein the negative control agents do not induce the one or more characteristics that are indicative of one or more taupathies or neurodegenerative conditions in a subjects;
one or more test agents, wherein the test agents may induce the one or more characteristics that are indicative of one or more taupathies or neurodegenerative conditions in the subject; and
one or more analytical instruments or techniques that detect the one or more characteristics that are indicative of one or more taupathies or neurodegenerative conditions in the subject, wherein a comparison of the test agents versus the positive and negative controls is indicative of having an effect on the cells.

2. The system of claim 1, wherein the one or more taupathies or neurodegenerative conditions are selected from the group consisting of Alzheimer's disease (AD), Parkinson's disease, Huntington's disease, frontotemporal dementia, progressive supranuclear palsy, corticobasal degeneration, and frontotemporal lobar degeneration (Pick's disease).

3. The system of claim 1, wherein the characteristics comprise formation of one or more neurofibrillary tangles (NFTs), tau protein aggregation, tau protein hyperphosphorylation, tau protein cleavage or any combinations thereof.

4. The system of claim 1, wherein the one or more positive control peptides comprise an amino acid sequence VQIVYK (SEQ. ID NO.: 1), VQIINK (SEQ. ID NO.: 2), VQVVYK (SEQ. ID NO.: 6), VQIVFK (SEQ. ID NO.: 7) or any combinations thereof and the one or more negative control peptides comprise an amino acid sequence VQIVKK (SEQ. ID NO.: 4), VQVVVK (SEQ. ID NO.: 5), or any combinations thereof.

5. The system of claim 1, wherein the subject is selected from the group consisting of a human subject, a rodent, a mouse, and a mammalian subject.

6. The system of claim 1, wherein the one or more analytical instruments or techniques comprise staining techniques, microscopy, aggregation kinetic analysis, immunochemical and Western Blot analysis, cell viability analysis, or any combinations thereof.

7. An in vitro cell culture method for detection of one or more characteristics indicative of Alzheimer's Disease (AD) and other taupathies in a subject suspected of having AD or the other taupathies comprising the steps of:
providing one or more cells or cell-cultures dispersed in a medium, wherein the one or more cells or cell cultures comprise neuronal cells lines, neuroblastomas, cerebellar granule neurons, glial cells, neuronal cultures, neurons, kidney fibroblasts, hippocampal cells, human embryonic kidney cells, or any combinations thereof;
providing one or more positive control agents, wherein the positive control agents induce the one or more characteristics that are indicative of AD and other taupathies;
providing one or more negative control agents, wherein the negative control agents do not induce the one or more characteristics that are indicative of AD and other taupathies;
obtaining a test sample from the subject suspected of having AD or the other taupathies, wherein the test sample is an isolated protein, a peptide, a biological sample, a tissue sample or any combinations thereof;
incubating the test sample, the positive control agent, and the negative control agent with the one or more cells or cell culture; and
detecting one or more characteristics indicative of AD and other taupathies in the one or more cells or cell culture following the incubation by a combination of staining techniques, microscopy, aggregation kinetic analysis, immunochemical and Western Blot analysis, and cell viability analysis.

8. The method of claim 7, wherein the presence of one or more neurofibrillary tangles (NFTs), tau protein aggregation, tau protein hyperphosphorylation, tau protein cleavage is indicative of AD and other taupathies.

9. The method of claim 7, wherein the other taupathies comprise frontotemporal dementia, progressive supranuclear palsy, corticobasal degeneration, and frontotemporal lobar degeneration (Pick's disease) or any combinations thereof.

10. The method of claim 7, wherein the one or more positive control peptides comprise an amino acid sequence VQIVYK (SEQ. ID NO.: 1), VQVVYK (SEQ. ID NO.: 6), VQIVFK (SEQ. ID NO.: 7) or any combinations thereof.

11. A method for screening and testing an activity of one or more agents against AD and other taupathies comprising the steps of:
providing one or more cells or cell-cultures dispersed in a medium, wherein the one or more cells or cell cultures comprise cerebellar granule neurons, glial cells, neuronal cultures, neurons, kidney fibroblasts, hippocampal cells, human embryonic kidney cells, or any combinations thereof;
providing one or more positive control agents, wherein the positive control agents induce the one or more characteristics that are indicative of AD and other taupathies;
providing one or more negative control agents, wherein the negative control agents do not induce the one or more characteristics that are indicative of AD and other taupathies;
providing one or more test agents against AD or the other taupathies, wherein the test agents comprise naturally occurring, synthesized or isolated proteins, peptide, chemical and biological substances or any combinations thereof;
incubating the test agent, the positive control agent, and the negative control agent with the one or more cells or cell culture; and
detecting one or more characteristics indicative of AD and other taupathies in the one or more cells or cell culture following the incubation by a combination of staining techniques, microscopy, aggregation kinetic analysis, immunochemical and Western Blot analysis, and cell viability analysis.

12. The method of claim 11, wherein the activity of the test agent is determined by an inhibition in a formation of one or more neurofibrillary tangles (NFTs) and an inhibition of tau protein aggregation, hyperphosphorylation, and cleavage in the one or more cells or cell culture.

13. The method of claim 11, wherein the other taupathies comprise frontotemporal dementia, progressive supranuclear palsy, corticobasal degeneration, and frontotemporal lobar degeneration (Pick's disease) or any combinations thereof.

14. The method of claim 11, wherein the one or more positive control peptides comprise an amino acid sequence VQIVYK (SEQ. ID NO.: 1), VQIVKK (SEQ. ID NO.: 4), VQVVVK (SEQ. ID NO.: 5), VQVVYK (SEQ. ID NO.: 6), VQIVFK (SEQ. ID NO.: 7) VQVVYK-R₉ (V-peptide) (SEQ. ID NO.: 6), VQVVVK-R₉ (VV-peptide) (SEQ. ID NO.: 5), VQIVKK-R₉ (K-peptide) (SEQ. ID NO.: 4), and VQIVFK-R₉ (F-peptide) (SEQ. ID NO.: 7), or any combinations thereof.

15. A pharmaceutical composition for treating AD or other taupathies in a subject comprising:
one or more active agents dispersed in a pharmaceutical carrier, wherein the one or more active agents inhibit a formation of one or more neurofibrillary tangles (NFTs) and inhibit tau protein aggregation, hyperphosphorylation, and cleavage in the subject; and
one or more optional excipients, fillers, diluents, extended or controlled release agents, bulking agents, antiadherents, binders, lubricants, preservatives or any combinations thereof.

16. The composition of claim 15, wherein the other taupathies comprise frontotemporal dementia, progressive supranuclear palsy, corticobasal degeneration, and frontotemporal lobar degeneration (Pick's disease) or any combinations thereof.

17. The composition of claim 15, wherein the one or more peptides comprise an amino acid sequence VQIVKK (SEQ. ID NO.: 4), VQVVVK (SEQ. ID NO.: 5), or any combinations thereof.

18. The composition of claim 15, wherein the composition is administered subcutaneously, intravenously, peritoneally, orally, and intramuscularly.

19. A method for treating AD and other taupathies in a subject comprising the steps of:
identifying the subject in need for treatment against AD and other taupathies; and
administering a pharmaceutical composition comprising one or more active agents dispersed in a pharmaceutical carrier, wherein the one or more active agents inhibit a formation of one or more neurofibrillary tangles (NFTs) and inhibit tau protein aggregation, hyperphosphorylation, and cleavage in the subject

20. The method of claim 19, further comprising the steps of:
monitoring the inhibition of the formation one or more neurofibrillary tangles (NFTs) and tau protein aggregation, hyperphosphorylation, and cleavage in the subject; and
administering the pharmaceutical composition repeatedly if necessary until the formation of one or more neurofibrillary tangles (NFTs) and tau protein aggregation, hyperphosphorylation, and cleavage in the subject is halted.

21. The method of claim 19, wherein the other taupathies comprise frontotemporal dementia, progressive supranuclear palsy, corticobasal degeneration, and frontotemporal lobar degeneration (Pick's disease) or any combinations thereof.

22. The method of claim 19, wherein the one or more active agents comprise naturally occurring, synthesized or isolated proteins, peptide, chemical and biological substances or any combinations thereof.

23. The method of claim 19, wherein the one or more peptides comprise an amino acid sequence VQIVKK (SEQ. ID NO.: 4), VQVVVK (SEQ. ID NO.: 5), or any combinations thereof.

24. The method of claim 19, wherein the composition is administered subcutaneously, intravenously, peritoneally, orally, and intramuscularly.

25. A composition for treating taupathies or neurodegenerative conditions in a subject comprising:
a fusion peptide comprising an amyloid forming peptide and a cytoplasmic penetration peptide, wherein the fusion protein triggers cell death.

26. The composition of claim 25, wherein the fusion peptide is selected from the group consisting of Ac-vqivyk-R₉—NH₂ (lower case amino acid codes denote D-amino acids) (SEQ. ID NO.: 1), Ac-vqivyk-NH₂ (Core peptide) (SEQ. ID NO.: 1), Ac-vqvvyk-R₉—NH₂ (V-peptide) (SEQ. ID NO.: 6), Ac-vqvvvkR₉—NH₂ (VV-peptide) (SEQ. ID NO.: 5), Ac-vqivkk-R₉—NH₂ (K-peptide) (SEQ. ID NO.: 4), Ac-VQIVYKR₉—NH₂ (T (L)-peptide) (SEQ. ID NO.: 1), and Ac-vqivfk-R₉—NH2 (F-peptide) (SEQ. ID NO.: 7).

27. The composition of claim 25, wherein the one or more taupathies or neurodegenerative conditions are selected from the group consisting of Alzheimer's disease (AD), Parkinson's disease, Huntington's disease, frontotemporal dementia, progressive supranuclear palsy, corticobasal degeneration, and frontotemporal lobar degeneration (Pick's disease).

28. An in vitro cell culture model or system for studying one or more characteristics that are indicative of one or more taupathies or neurodegenerative conditions in a subject comprising:
one or more cells or cell-cultures dispersed in a medium, wherein the one or more cells comprise cerebellar granule neurons, glial cells, neuronal cultures, neurons, kidney fibroblasts, hippocampal cells, human embryonic kidney cells, or any combinations thereof;
a fusion peptide comprising an amyloid forming peptide and a cytoplasmic penetration peptide, wherein the fusion protein triggers aggregation and neuronal cell death; and
one or more test agents, wherein the test agents may cause survival of the neuronal cells by the fusion peptide.

29. The system of claim 28, wherein the one or more taupathies or neurodegenerative conditions are selected from the group consisting of Alzheimer's disease (AD), Parkinson's disease, Huntington's disease, frontotemporal dementia, progressive supranuclear palsy, corticobasal degeneration, and frontotemporal lobar degeneration (Pick's disease).

30. The system of claim 28, wherein the fusion protein comprises a fusion peptide selected from VQVVYK-R₉ (V-peptide) (SEQ. ID NO.: 6), VQVVVK-R₉ (VV-peptide) (SEQ. ID NO.: 5), VQIVKK-R₉ (K-peptide) (SEQ. ID NO.: 4), and VQIVFK-R₉ (F-peptide) (SEQ. ID NO.: 7), or any combinations thereof.


SEQUENCE LISTING (10 sequences)

SEQ ID NO: 1 - protein, 6 aa, homo sapiens
VQIVYK

SEQ ID NO: 2 - protein, 6 aa, homo sapiens
VQIINK

SEQ ID NO: 3 - protein, 6 aa, homo sapiens
KLVFFA

SEQ ID NO: 4 - protein, 6 aa, homo sapiens
VQIVKK

SEQ ID NO: 5 - protein, 6 aa, homo sapiens
VQVVVK

SEQ ID NO: 6 - protein, 6 aa, homo sapiens
VQVVYK

SEQ ID NO: 7 - protein, 6 aa, homo sapiens
VQIVFK

SEQ ID NO: 8 - protein, 5 aa, homo sapiens
KLTFR

SEQ ID NO: 9 - protein, 6 aa, homo sapiens
STVIIE

SEQ ID NO: 10 - protein, 6 aa, homo sapiens
SQIVYA